SEQUENCE LISTING (9 sequences)

SEQ ID NO: 1 - DNA, 25 bp, Artificial Sequence
  Sense PCR primer
ctgaattctttcggatcgaagggtg

SEQ ID NO: 2 - DNA, 42 bp, Artificial Sequence
  Antisense polyA PCR primer
tttttttttttttttttttttttactcttccatttgtttccc

SEQ ID NO: 3 - DNA, 58 bp, Artificial Sequence
  Antisense polyA  PCR primer
tttttttttttttttttttttttttttttttttttttttactcttccatttgtttccc

SEQ ID NO: 4 - DNA, 42 bp, Artificial Sequence
  Antisense polyT PCR primer
aaaaaaaaaaaaaaaaaaaaaatactcttccatttgtttccc

SEQ ID NO: 5 - DNA, 25 bp, Artificial Sequence
  oligo(dt) biotinylated
ttttttttttttttttttttttttt

SEQ ID NO: 6 - DNA, 25 bp, Artificial Sequence
  Sense PCR primer
ctgaattcgggtacgtaacggatct

SEQ ID NO: 7 - DNA, 40 bp, Artificial Sequence
  Antisense PolyA PCR primer
tttttttttttttttttttttttactgcggcattcttctg

SEQ ID NO: 8 - DNA, 29 bp, Artificial Sequence
  Sense PCT primer
ctgaattcctttaaacggttcggcacacg

SEQ ID NO: 9 - DNA, 43 bp, Artificial Sequence
  Antisense polyA PCR primer
tttttttttttttttttttttttcgtgctgtgacatatagctg

(19) United States
(12) Patent Application Publication
(10) Pub. No.: US 2012/0135417 A1
(43) Pub. Date: May 31, 2012

(54) METHODS FOR SCREENING AND IDENTIFYING COMPOUNDS

(76) Inventors: Jayhyuk Myung, Woodbridge, CT (US); Kenneth F. Blount, Guilford, CT (US); Christen Douglas Forbes, North Haven, CT (US); David Osterman, Glastonbury, CT (US)

(21) Appl. No.: 13/375,865

(22) PCT Filed: Jun. 2, 2010
(86) PCT No.: PCT/US10/01611

§ 371 Date: Dec. 2, 2011

Related U.S. Application Data
(60) Provisional application No. 61/183,166, filed on Jun. 2, 2009.

(51) Int. Cl. C12Q 1/68

(52) U.S. Cl. 435/6.12; 435/6.13

(57) ABSTRACT

Methods, compositions and assays that measure the effect of a test compound on induction of ligand-induced ribowitch-mediated transcription termination are disclosed. The methods and the assays are useful in identifying drug candidates that modulate transcription by binding to a riboswitch, for example.

This application claims the benefit of U.S. Provisional Application No. 61/183,166, filed Jun. 2, 2009, the contents of which are incorporated herein by reference.

### FIELD OF THE INVENTION

The field relates to the biochemistry, molecular biology, and biochemical pharmacology of riboswitch-based genetic control elements. The field also relates to methods and compositions for evaluating riboswitch-mediated transcription processes. The field also relates to the screening and identifying of compounds that may be used for the treatment of bacterial, fungal, and other human and veterinary infectious diseases, as well for other research and development, agricultural, and industrial applications where modulation of gene expression by riboswitch ligands is desirable.

### BACKGROUND OF THE INVENTION

The identification of small molecules that target and affect crucial steps in cellular pathways is a first step in the process of drug discovery. To accomplish this goal, assays may be developed and configured to reflect the biological activity of a prospective drug target that is critical for a given pathway. In addition, these assays should be reliable and give a robust signal of the biological activity. When such assays are amenable to high throughput screening (HTS), the effort to find novel chemical matter that can serve as a starting point for drug development may be greatly accelerated.

In many bacteria, RNA structures termed riboswitches regulate the expression of various genes crucial for survival or virulence. Typically located within the 5′-untranslated region (5′-UTR) of certain mRNAs, members of each known class of riboswitch can fold into a distinct, three-dimensionally structured receptor that recognizes a specific organic metabolite. When the cognate metabolite is present at sufficiently high concentrations during transcription of the mRNA, the riboswitch receptor binds to the metabolite and induces a structural change in the nascent mRNA that prevents expression of the open reading frame (ORF), either by inducing transcription termination prior to formation of a full-length transcript or by preventing ribosomes from initiating translation of the ORF. In the absence of the cognate metabolite, the riboswitch folds into a structure that does not interfere with the expression of the ORF.

Seventeen different classes of riboswitches have been reported. Members of each class of riboswitches bind to the same metabolite and share a highly conserved sequence and secondary structure. Riboswitch motifs have been identified that bind to thiamine pyrophosphate (TPP), flavin mononucleotide (FMN), glycine, adenine, guanine, 3′-5′-cyclic diguanylic acid (c-di-GMP), molybdenum cofactor, glucosamine-6-phosphate (GlcN6P), lysine, adenine, and adenosylcobalamin (AdoCbl) riboswitches. Additionally, four distinct riboswitch motifs have been reported that recognize S-adenosylmethionine (SAM) and two distinct motifs that recognize pre-queuosine-1 (PreQ1).

Riboswitch receptors bind to their respective ligands in an interface that approaches the level of complexity and selectivity of proteins. This highly specific interaction allows riboswitches to discriminate against most intimately related analogues of ligands. For instance, the receptor of a guanine-binding riboswitch from Bacillus subtilis forms a three-dimensional structure such that the ligand is almost completely enveloped. The guanine is positioned between two aromatic bases and each polar functional group of the guanine forms hydrogen bonds with four additional riboswitch nucleotides surrounding it. This level of specificity allows the riboswitch to discriminate against most closely related purine analogs. Similarly, studies of the SAM-binding riboswitches reveal that nearly every functional group of SAM is critical for the binding interaction, allowing the riboswitch to differentiate highly similar compounds such as S-adenosylhomocysteine (SAH) and S-adenosylmethionine (SAM), which differ by a single methyl group. Likewise, TPP riboswitches comprise one subdomain that recognizes every polar functional group of the 4-amino-5-hydroxymethyl-2-methylpyrimidine (HMP) moiety, albeit not the thiazole moiety, and another subdomain that coordinates two metal ions and several water molecules to bind the negatively charged pyrophosphate moiety of the ligand. Similar to TPP, guanine, and SAM riboswitches, FMN riboswitches form receptor structures that are highly specific for the natural metabolite FMN.

The highly specific interaction that riboswitches have with their cognate ligands presents an opportunity for the design of highly-selective small molecules that could, in principle, lead to the repression of specific genes. Indeed, several antibacterial antimetabolite ligands have already been identified that bind to known riboswitch classes, including pyrithiamine pyrophosphate (PTPP) which binds TPP riboswitches, L-aminoethylcysteine (AEC) and DL-4-oxalysine which bind to lysine riboswitches and roseoflavin which binds to FMN riboswitches. In each case, the metabolite mimics triggers riboswitch-mediated repression of a gene or genes important to bacterial survival and thereby prevents bacterial growth.

For instance, roseoflavin, a natural analogue of riboflavin originally isolated from Streptomyces davawensis and shown to have antimicrobial activity (Matsui, K., Wang, H., Hirota, T., Matsukawa, H., Kasai, S., Shinagawa, K., and Otani, S., 1982 Agric. Biol. Chem. 46, 2003-2008) was shown to bind to FMN riboswitches and modulate the expression of genes involved in riboflavin production and transport. Lee et al. demonstrated that roseoflavin represses the expression of an FMN riboswitch-regulated β-galactosidase reporter gene in wild-type B. subtilis cells (Lee, E. R., Blount, K. F., and Breaker, R. R., 2009 RNA Biology 6, 187-194). Sequence analysis of previously identified B. subtilis and Lactococcus lactis mutants that are resistant to roseoflavin indicated that many of the resistance mutations map to the FMN riboswitch that regulates the ribDEAHT operon (Burgess, C., O'Connel-Motherway, M., Sybesma, W., Hugenholtz, J., and van Sinderen, D., 2004, Appl. Environ. Microbiology 70, 5769-5777, Kreneva, R. A. and Perumov, D. A. 1990 Mol. Gen. Genet. 222, 467-469, Kil, Y. V., Mironov, V. N., Gorishin, I., Kreneva, R. A., and Perumov, D. A. 1992, Mol. Gen. Genet. 233, 483-486). Moreover, engineering of these mutations into riboswitch sequences disrupts ligand binding in vitro and derepresses the expression of an FMN riboswitch-regulated reporter gene inside B. subtilis (Lee, E. R., Blount, K. F., and Breaker, R. R., 2009 RNA Biology 6, 187-194).

In a similar example, the antibacterial and antifungal thiamine analog pyrithiamine (PT) has been shown to exert its growth inhibitory effect by targeting thiamine pyrophosphate-binding riboswitches and thereby repressing the expression of thiamine biosynthesis genes. PT is phosphorylated in cells to pyrithiamine pyrophosphate (PTPP) (Iwashiman, A., Wakbabayashi, Y., and Nose, Y., 1976 J. Biochem. (Tokyo) 79, 845-847, Elnageh, K. M. and Zia-ur-Rahman, N. A. 2001 Int. J. Aric. Biol. 3, 178-180). PTPP binds in vitro to several TPP riboswitches with binding affinities nearly identical to that of TPP, and thiamine and PT added to the cultures are each able to reduce expression of a TPP riboswitch-regulated β-galactosidase expression in transgenic B. subtilis and E. coli (Sudarsan, N., Cohen-Chalamish, C., Nakamura, S., Emilsson, G. M., and Breaker, R. R., 2005 Chemistry and Biology 12, 1325-1335). This suggests that PTPP competes with TPP for binding to TPP riboswitch in the cells and may inhibit bacterial or fungal growth by interfering with TPP-regulated gene expressions. Indeed, bacteria selected for PT resistance contains mutations that disrupt both TPP and PTPP binding.

In yet another example, Lysine analogs, L-aminoethylcystein (AEC) and DL-4-oxalysine, inhibit growth of certain Gram-positive bacteria (Shiota, T, Folk, J. E., and Tietze, F. 1958 Arch. Biochem. Biophys. 77, 372-377, McCord, T., Ravel, J., Skinner, C., and Shive, W., 1957 JACS 79, 5693-5696) at least in part through targeting lysine riboswitches that regulate lysine precursor biosynthesis (Blount, K. F., Wang, X. J., Lim, J., Sudarsan, N., and Breaker, R. R. 2007, Nat. Chem. Bio. 3, 44-49, Sudarsan, N., Wickiser, J. K., Nakamura, S., Ebert M. S., and Breaker, R. R., 2003 Genes Dev. 17, 2688-2697, Lu, Y, Shevtcheniko, T., and Paulus, H. 1992 FEMS Microbiol. Lett. 71, 23-27).

The standard methodology for assessing the effects of a potential ligand on riboswitch-mediated transcription processes uses an in vitro polyacrylamide gel-based method (Winkler, W. C., Cohn-Chalamish, S., and Breaker, R. R., 2002 PNAS USA 99, 15908-15913, Wickiser, J. K., Winkler, W. C., Breaker, R. R., and Crothers, D. M., 2005 Mol. Cell 18, 49-60). In this assay, in vitro transcription reactions are performed by incubating purified RNA polymerases from E. coli or B. subtilis in the presence of Mg²⁺ and ribonucleoside triphosphates (rNTPs) with the DNA sequence that carries a promoter for initiating transcription, linked to a riboswitch aptamer domain, and a riboswitch expression platform, which controls transcription of a coding region encoding a downstream nucleotide domain. [α-³²P] ATP is added to the transcription reaction to radiolabel the transcription product. A potential ligand is added to the reaction mix and transcription is allowed to occur. After a suitable amount of time, the reaction is stopped and the full-length and truncated transcripts are separated using polyacrylamide gel electrophoresis (PAGE). Relative amounts of the transcripts are quantitated by autoradiography. In the case of the FMN riboswitch, among others, an active ligand will increase the proportion of truncated transcript. This is manifested by an increase in the relative amount of a shorter, faster-migrating band on the gel. This method suffers from the labor intensive process of setting up, running, and analyzing the gels. Throughput for this assay is very limited and would preclude the testing of hundreds or thousands of novel compounds, which is a part of modern drug discovery. Highly desirable would be an assay format incorporating microtiter plates to facilitate the screening of many compounds in parallel.

A plate-based assay has been described for assessing activity possessed by a certain atypical riboswitch termed the glmS riboswitch (Breaker, R. R., Blount, K. F., Puskarz, I. J., and Wickiser, J. K., WO 2007/100412, Blount, K., Puskarz, L, Penchovsky, R., and Breaker, R., 2006 RNA Biol., 3, e1-e5, Mayer, G. and Famulok 2006 Chem. Bio. Chem. 7, 602-604). This assay relies for detection on ribozyme activity that is specific to this particular riboswitch. A ribozyme (the term is derived from ribonucleic acid enzyme) is an RNA molecule that catalyzes a chemical reaction, in this case the hydrolysis of a phosphodiester bond. As a result, this assay is not applicable to other known naturally occurring riboswitches, as the method is not able to assess whether or not a ligand induces transcription termination. In addition, a fluorescent-ligand displacement assay has been disclosed by Breaker et al (Breaker, R. R., Blount, K. F., Puskarz, I. J., and Wickiser, J. K., WO 2007/100412). However, this ligand displacement assay is limited in its use since it only measures binding events that may not cause a functional activity of the riboswitch. Thus, there remains a need for improved assays to identify small molecule compounds that bind to a riboswitch and deactivate or prevent the transcription process under the control of the riboswitch. There also remains a need for assays that are applicable to measure activity for a wider variety of riboswitches as well as to evaluate riboswitch function in a high throughput manner.

### SUMMARY OF THE INVENTION

In one example, a method for measuring the effect of a test compound on induction of riboswitch-mediated transcription termination comprises:

providing a DNA sequence having a promoter operably linked to a riboswitch aptamer domain, and a riboswitch expression platform, which controls transcription of a coding region encoding a signaling sequence;

incubating, in the presence or absence of a test compound, the DNA sequence with an RNA polymerase and a plurality of ribonucleotides, at least some of which are labeled, to express a RNA product; capturing the RNA product having a signaling sequence, using a capture element which specifically recognizes at least a portion of the signaling sequence, wherein the capture element is bound to a substrate;

removing uncaptured products; and

detecting the signal of the signaling sequence, wherein a decrease or absence of signal of the signaling sequence in the presence of a test compound relative to the signal in the absence of a test compound indicates ligand-induced riboswitch-mediated transcription termination.

In one example, a DNA sequence having a promoter operably linked to a riboswitch aptamer domain, and a riboswitch expression platform domain, which controls transcription of a coding region encoding a signaling sequence is incubated with an RNA polymerase and a plurality of ribonucleoside triphosphates, wherein one of the plurality of ribonucleoside triphosphates is radiolabeled, an example of labeling. In one example, the RNA polymerase is a purified sigma-rich E. coli RNA polymerase. In one example, the signaling sequence is a polyA sequence and the capture element is a deoxythymidylate oligonucleotide. In the capturing step, an immobilized or immobilizable capture element comprising solely of deoxythymidylates (oligo (dT)) hybridizes to the full-length RNA product having a polyA sequence via base-pairing between the deoxythymidylate and the adenylate. The capture element, in one example, is tagged with biotin. In another example, the capture element is an antibody.

If a capture element is a biotinylated oligonucleotide, the biotinylated capture element may subsequently be immobilized by binding to a streptavidin-coated plate via biotin-streptavidin interaction, for example. In the presence of a riboswitch binding compound such as FMN, a truncated RNA transcript lacking a polyA sequence is produced. Thus, this truncated RNA transcript would not be captured by a corresponding capture element that normally would hybridize to the polyA sequence-containing RNA product. In the detecting step, a decrease in signal of the polyA sequence would be noticed in the presence of compounds that bind to a riboswitch, as lesser amounts of full-length RNA transcript would be generated. Thus, the methods may readily identify small molecule compounds that bind to a riboswitch and deactivate or prevent the transcription process under the control of the riboswitch.

The methods and the assays as disclosed may also be applied to the use of a streptavidin-coated bead as a substrate, rather than the use of a streptavidin-coated plate.

The methods and the assays also utilize a streptavidin-coated SPA bead alternatively as a substrate. The SPA bead is a scintillant-impregnated bead that emits light when a substance bound to the bead experiences a radioisotope decay event. The use of such a bead would eliminate the need for washing the plate to remove unincorporated radioisotope, making this a homogeneous assay, and increasing the simplicity and speed of execution of the assay.

The methods and the assay may also utilize a streptavidin-coated FlashPlate as a substrate. The FlashPlate contains an impregnated scinitillant. This method would eliminate the need to wash plates to remove unincorporated radioisotope.

In another example, a method for measuring the effect of a test compound on induction of riboswitch-mediated transcription termination, comprises:
providing a DNA sequence having a promoter operably linked to a riboswitch aptamer domain, and a riboswitch expression platform domain, which control transcription of a coding region encoding a signaling sequence; incubating, in the presence or absence of a test compound, the DNA sequence with an RNA polymerase and a plurality of ribonucleotides, to express an RNA product;
capturing the RNA product having a signaling sequence, using a capture element which specifically recognizes at least a portion of the signaling sequence, wherein the capture element is bound to a substrate;
attaching a detection element to the captured RNA product wherein the detection element specifically recognizes at least a portion of the non-captured portion of the RNA product; removing uncaptured products; and
detecting the signal generated by the detection element, wherein a decrease or absence of signal of the signaling sequence, in the presence of a test compound relative to the signal in the absence of a test compound indicates ligand-induced riboswitch-mediated transcription termination.

In one example, following the capture of the full-length transcript, a detection element complementary to a portion of the transcript not involved in the capture may be hybridized to the captured product and used for signal generation and amplification. For example, the portion of the transcript not involved in the capture may be any sequence region excluding the polyA sequence, such as the riboswitch sequence. If the detection element is radiolabeled, for example, the amount of the detection element attached to the captured RNA product may be quantitated by measuring radioactivity. In another example, if the detection element is a fluorescently-labeled probe, the amount of the detection element attached to the captured RNA products may be quantitated by measuring fluorescence signal intensity.

If the detection element is tagged with biotin, then the amount of this biotinylated detection element may be quantitated and its signal amplified by using streptavidin-conjugated horseradish peroxidase (HRP) and a peroxidase substrate, for example. Streptavidin-conjugated HRP binds to the biotinylated detection element which is attached to the captured RNA product via streptavidin-biotin interaction. After unbound streptavidin-conjugated HRP is removed by washing, HRP substrate is then added for its HRP-catalyzed conversion to a product. For example, a non-fluorescent HRP substrate is used and may be converted by HRP to a fluorescent product. Alternatively, a fluorescent HRP substrate is used and may be converted by HRP to a non-fluorescent product.

Other examples of quantification and signal amplification may be used for the detection element. For example, a detection element is an oligonucleotide that comprises FRET (fluorescence energy transfer) pairs consisting of donor and acceptor molecules. This detection element may be designed to hybridize to a portion of the RNA transcript not involved in the capture. The portion of the RNA transcript not involved in the capture may be any sequence region excluding the polyA sequence. In addition, this FRET-pair-containing oligonucleotide is designed to form a hairpin structure to allow fluorescence energy transfer between donor and acceptor molecules when it is free from the captured full-length transcript. Upon hybridization of this FRET-pair-containing oligonucleotide to the captured RNA products, the FRET-pair-containing oligonucleotide is designed to lose the hairpin structure, thus separating the acceptor and donor molecules and eliminating the FRET signal.

Alternatively, a fluorophore-fused detection element may be used as a fluorescence polarization probe. This detection element may be an oligonucleotide, for example. The detection element is designed to bind to the portion of RNA transcript not involved in the capture. The fluorophore is excited with polarized light. The resulting emission is captured using both parallel and perpendicular polarized filters. A rapidly tumbling fluorophore (“unbound”) will rotate during the fluorescence lifetime and thus show significant emission in the perpendicular channel. A molecule attached to a slowly rotating macromolecule (“bound”) will show almost all the emission in the parallel channel. Thus,the fluorophore-fused detection element hybridizing to the portion of the RNA product not involved in the capture will show significant emission in the parallel channel upon excitation of the fluorophore with polarized light. By contrast, unbound fluorophore-fused detection element would show significant emission in the perpendicular channel.

To evaluate the binding of a small molecule compound and its effect on induction of riboswitch-mediated gene expression, the concentration of the compound may be varied among a number of parallel in vitro transcription reactions.

In another example, a screening assay for drug candidates targeting riboswitches comprises: any of the methods described.

The methods and the assays may be applied to any riboswitch that modulates the balance between full-length and truncated transcription. A DNA sequence carrying a riboswitch motif and additional nucleotide sequence that encompasses the putative terminator and antiterminator elements and contains a segment that may be captured is incubated in a reaction mixture with a RNA polymerase and rNTPs. Only the full length transcript would be captured and test compounds that significantly alter the amount of full-length transcript would be identified as “hits.”

These methods and the assays may identify and distinguish small molecule compounds that bind to a riboswitch and deactivate or prevent the transcription process under the control of the riboswitch. The methods and compositions are amenable to high throughput screening (HTS), unlike many existing technologies, and may be applied currently to the testing of compound libraries for compounds that productively bind to riboswitches.

The methods and the assays may be used to identify compounds for antibacterial and antifungal chemotherapy, as well for other research and development, and agricultural and industrial applications where modulation of gene expression by riboswitch ligands is desirable.

One advantage is that the methods and the assays are simpler and thus less consuming of time and materials.

Another advantage is that the methods and the assays are amenable to high throughput screening and may be used to test large compound libraries.

Yet another advantage is that the methods and the assays measure the functional outcome of ligand binding to the riboswitch, not just a binding event that may or may not cause a change in the functional activity of the riboswitch.

Still another advantage is that the methods and the assays may be designed to generically apply to any riboswitch that modulates the balance between full-length and truncated transcription.

### BRIEF DESCRIPTION OF THE FIGURES

FIG. 1 shows a schematic diagram of ligand-induced riboswitch-mediated transcription regulation using rib leader sequence derived from the ribDEAHT operon of B subtilis, responsive to FMN.

FIG. 2 shows a schematic diagram of ligand-induced riboswitch-mediated transcription regulation, responsive to TPP.

FIG. 3 shows a schematic diagram of ligand-induced riboswitch-mediated transcription regulation, responsive to SAM.

FIG. 4 depicts a schematic diagram of one embodiment of the method.

FIG. 5A shows a detection of in vitro transcription product, measured in cpm. Black column representing full length transcript in absence of FMN, grey column is in presence of FMN. Columns on left show experiment using polyA tail. Columns on right is a control experiment using a poly T tail.

FIG. 5B depicts signal dependence on biotinylated oligo (dT). Black columns are absence of FMN, grey are in presence of 100 micromolar FMN. As concentration of biotinylated oligo dT capture element increases, signal increases, then levels off at saturation.

FIG. 6 shows a graph of signal differences with and without FMN at varying concentrations of magnesium and rNTPs.

FIG. 7 shows a graph of signals in absence (black) and presence (100 micromolar FMN, grey), with fixed amount of capture element and varying concentration of DNA sequence.

FIG. 8A shows an example of a titration of RNA polymerase concentrations in one time course experiment in the absence of FMN.

FIG. 8B depicts another example of a titration of RNA polymerase in a time course experiment in the presence of FMN.

FIG. 9 shows a graph of percentage of activity versus pH.

FIG. 10 depicts a graph of inhibition of E. coli polymerase activity by rifampin.

FIG. 11 demonstrates screening results for FMN and FMN analogs.

FIG. 12 demonstrates screening results for TPP and TPP analogs.

FIG. 13 demonstrates screening results for TPP and TPP analogs.

FIG. 14 demonstrates screening results for FMN and FMN analogs.

FIG. 15 demonstrates screening results for TPP and TPP analogs.

FIG. 16 demonstrates screening results for TPP and TPP analogs.

### DETAILED DESCRIPTION

The examples and drawings provided in the detailed description are merely examples, which should not be used to limit the scope of the claims in any claim construction or interpretation.

In one example, Method I measures the effect of a test compound on induction of riboswitch-mediated transcription termination, comprising:
providing a DNA sequence having a promoter operably linked to a riboswitch aptamer domain, and a riboswitch expression platform, which controls transcription of a coding region encoding a signaling sequence;
incubating, in the presence or absence of a test compound, the DNA sequence with an RNA polymerase and a plurality of ribonucleotides, at least some of which are labeled, to express an RNA product;
capturing the RNA product having a signaling sequence, using a capture element which specifically recognizes at least a portion of the signaling sequence; wherein the capture element is bound to a substrate;
removing uncaptured products; and detecting the signal of the signaling sequence, wherein a decrease or absence of signal of the signaling sequence in the presence of a test compound relative to the signal in the absence of a test compound indicates ligand-induced riboswitch-mediated transcription termination.
   1.1 Method I, wherein the signaling sequence is a defined nucleotide sequence and the capture element is an oligonucleotide complementary to the signaling sequence. A defined nucleotides is one known to a person of ordinary skill in the art that may be used as a signaling sequence. An example of a defined nucleotide sequence is a polyA sequence, for example.
   1.2 Method I or 1.1, wherein the signaling sequence is a polyA sequence and the capture element is a deoxythymidylate oligonucleotide.
   1.3 Method of Method I, or 1.1 or 1.2, wherein at least one of the plurality of ribonucleotides is radiolabeled.
   1.4 Method of Method I, or 1.1, 1.2, or 1.3, wherein at least one of the plurality of ribonucleotides is labeled by having a radiolabeled ribonucleoside triphosphate.
   1.5 Method of Methods 1.3, or 1.4, wherein the step of detecting the signal of the signaling sequence includes measuring radioactivity, wherein a decrease in the signal or an absence of the signal indicates ligand-induced riboswitch-mediated transcription termination. For example, a scintillation counter may be used to measure radioactivity.
   1.6 Method of Method I, or 1.1 or 1.2, wherein at least one of the plurality of ribonucleotides is fluorescently labeled.
   1.7 Method of Method 1.6, wherein the step of detecting the signal of the signaling sequence includes measuring fluorescence intensity, wherein a decrease in the signal or an absence of the signal indicates ligand-induced riboswitch-mediated transcription termination.
   1.8 Method of any of Method I, or Methods 1.1-1.7, wherein the step of capturing includes providing the capture element which is the oligonucleotide complementary to the signaling sequence and the step includes hybridizing the RNA product to the oligonucleotide by complementary base pairing.
   1.9 Method of any of Method I, or Methods 1.3-1.7 wherein the step of capturing the RNA product with the capture element utilizes affinity binding.
   1.10 Method of Method 1.9, wherein the capture element is an antibody.
   1.11 Method of any of Method I, or Methods 1.1-1.10, wherein a capture element is biotinylated.
   1.12 Method of Method 1.11, wherein the capture element is immobilized by binding to a streptavidin-coated plate.
   1.13 Method of Method 1.11, wherein the capture element is immobilized using a streptavidin-coated Flash plate that includes an impregnated scintillant.
   1.14 Method of Method 1.11, wherein the capture element is immobilized using a streptavidin-coated bead.
   1.15 Method of Method 1.11, wherein the capture element is immobilized by using a Streptavidin-coated SPA bead.
   1.16 Method of Method I, or Methods 1.1-1.15, wherein the test compound is FMN or an analogue thereof.
   1.17 Method of Method I, or Methods 1.1-1.16, wherein a compound is being tested against any of the disclosed riboswitches. The disclosed riboswitches herein are any disclosed in the specification.
   1.18 Method of Method I, or Methods 1.1-1.17, wherein the method may be performed in a high throughput manner.
   1.19 Method of Method I, or Methods 1.1-1.18, wherein the step of incubating the DNA sequence includes providing an appropriate concentration of magnesium to modulate the speed of the riboswitch-mediated transcription reactions.
   1.20 Method of Method I, or Methods 1.1-1.19, wherein the step of incubating the DNA sequence includes adjusting a pH in order to modulate riboswitch-mediated transcription reactions.
   1.21 Method of Method I, or Methods 1.1-1.20, wherein the incubation and capture steps are carried out in a pH range from 6-10.
   1.22 Method of Method I, or Methods 1.1-1.21, wherein the step of incubating the DNA sequence is optimized by a preceding step of varying a concentration of the DNA sequence among a plurality of parallel in vitro transcription reactions.
   1.23 Method of Method I, or Methods 1.1-1.22, wherein the test candidate is an anti-bacterial drug candidate.
   1.24 Method of Method I, or Methods 1.1-1.23, wherein the test candidate is a gene expression regulator.
   1.25 Method of Method I, or any of Methods 1.1-1.24, wherein the incubation step is conducted in a pH range depending on the type of RNA polymerase utilized.
   1.26 Method of Method I or any of Methods 1.1-1.25, wherein the riboswitch sequence is a naturally occurring riboswitch sequence.
   1.27 Method of Method I, or any of Methods 1.1-1.26, wherein the riboswitch sequence is an engineered riboswitch sequence.
   1.28 Method of Method 1.1-1.25, wherein the riboswitch aptamer domain is engineered aptamer domain and the riboswitch expression platform is an engineered expression platform domain, which controls transcription of a coding region encoding a signaling sequence.

High throughput screening assays may be developed in which a test compound may be used to show a ligand-induced riboswitch-mediated transcription termination. Test compounds may be tested for their ability to bind to a riboswitch and thereby be able to induce riboswitch-mediated transcription termination.

The following definitions are provided from a related application, WO 2004/027035, the disclosure of which is hereby incorporated by reference.

A “promoter” is generally a sequence or sequences of DNA that function when in a relatively fixed location with regard to the transcription start site. A promoter contains core elements required for basic interaction of RNA polymerase and transcription factors and can contain upstream elements and response elements.

“Riboswitch aptamer domains” are nucleic acid segments and structures that can bind selectively to particular compounds and classes of compounds. Riboswitches have aptamer domains that, upon binding of a trigger molecule confer a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

“Expression platform domains” are a part of the riboswitch that affects expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, transcription antiterminators, and stability and processing signals.

In one example, Method II provides a method for measuring the effect of a test compound on induction of riboswitch-mediated transcription termination, comprising: providing a DNA sequence having a promoter operably linked to a riboswitch aptamer domain, and a riboswitch expression platform domain, which control transcription of a coding region encoding a signaling sequence;
incubating, in the presence or absence of a test compound, the DNA sequence with an RNA polymerase and a plurality of ribonucleotides, to express an RNA product;
capturing the RNA product having a signaling sequence, using a capture element which specifically recognizes at least a portion of the signaling sequence, wherein the capture element is bound to a substrate;
attaching a detection element to the captured RNA product; wherein the detection element specifically recognizes at least a portion of the non-captured portion of the RNA product;
removing uncaptured products; and
detecting the signal generated by the detection element, wherein a decrease or absence of signal of the signaling sequence, in the presence of a test compound relative to the signal in the absence of a test compound indicates ligand-induced riboswitch-mediated transcription termination.
   2.1 Method of Method II, wherein the signaling sequence is a defined nucleotide and the capture element is a complementary oligonucleotide. A defined nucleotide is one known to a person of ordinary skill in the art that may be used as a signaling sequence. For example, one example of a defined nucleotide is a poly A sequence.
   2.2 Method of Method II or 2.1, wherein the signaling sequence is a polyA sequence and the capture element is a deoxythymidylate oligonucleotide.
   2.3 Method of Method II, or Methods 2.1, or 2.2, wherein the step of hybridizing the RNA product to the capture element relies on complementary base pairing.
   2.4 Method of Method II, wherein the step of attaching the capture element to the RNA product utilizes affinity binding.
   2.5 Method of Method II, or Method 2.4, wherein the capture element is an antibody.
   2.6 Method of Method H, or Methods 2.1-2.5, wherein the step of attaching the detection element to the captured RNA product utilizes affinity binding.
   2.7 Method of Method II or Methods 2.1-2.5, wherein the detection element is an oligonucleotide complementary to a portion of the non-captured portion of the RNA product. For example, the non-captured portion is a portion of the RNA product that is not involved in the base pairing between the signaling sequence of the RNA product and the capture element.
   2.8 Method of Method 2.7, wherein the step of attaching the detection element to the captured RNA product relies on complementary base pairing.
   2.9 Method of Method II, or Methods 2.1-2.8, wherein the capture element is biotinylated.
   2.10 Method of Method II or Method of Method 2.9, wherein the capture element is immobilized by binding to a streptavidin-coated plate.
   2.11 Method of Method II or Method of Method 2.9, wherein the capture element is immobilized using a streptavidin-coated Flash plate that includes an impregnated scintillant.
   2.12 Method of Method H or Method of Method 2.9, wherein the capture element is immobilized using a streptavidin-coated bead.
   2.13 Method of Method II or Method of Method 2.9, wherein the capture element is immobilized by using a streptavidin-coated SPA bead.
   2.14 Method of Method II, or Methods 2.1-2.13, wherein the detection element is a labeled probe.
   2.15 Method of Method II, or Methods 2.1-2.14, wherein the detection element is a radiolabeled probe.
   2.16 Method of Method II or Methods 2.15, wherein the step of detecting the signal of the signaling sequence includes measuring radioactivity, wherein a decrease in the signal or an absence of the signal indicates a ligand-induced riboswitch-mediated transcription termination.
   2.17 Method of Method II, or Methods 2.1-2.14, wherein the detection element is a fluorescently labeled probe.
   2.18 Method of Method II, or Methods 2.1-2.14 or 2.17, wherein the step of detecting the signal of the signaling sequence includes measuring fluorescence intensity, wherein a decrease in the signal or an absence of the signal indicates a ligand-induced riboswitch-mediated transcription termination.
   2.19 Method of Method II, or Methods 2.1-2.13, wherein the detection element is capable of binding to a labeled probe.
   2.20 Method of Method II, or Methods 2.1-2.13 or 2.19, wherein the detection element is capable of binding to a radiolabeled probe.
   2.21 Method of Method II, or Methods 2.1-13 or 2.19-2.20, wherein the step of detecting the signal of the signaling sequence includes measuring radioactivity, wherein a decrease in the signal or an absence of the signal indicates a ligand-induced riboswitch-mediated transcription termination.
   2.22 Method of Method II, or Methods 2.1-2.13, wherein the detection element is capable of binding to a fluorescently labeled probe.
   2.23 Method of Method II, or Method 2.22, wherein the step of detecting the signal of the signaling sequence includes measuring fluorescence intensity, wherein a decrease in the signal or an absence of the signal indicates a ligand-induced riboswitch-mediated transcription termination.
   2.24 Method of Method II, or Methods 2.1-2.13, wherein the detecting the signal utilizes streptavidin-conjugated horseradish peroxide (HRP).
   2.25 Method of Method II, or Methods 2.1-2.13, or 2.24, wherein the detecting the signal utilizes a horseradish peroxidase (HRP) substrate.
   2.26 Method of Method II, or Methods 2.25, wherein the HRP substrate is a non-fluorescent HRP substrate that may be converted to a fluorescent product.
   2.27 Method of Method II, or Methods 2.25, wherein the HRP substrate is a fluorescent HRP substrate that may be converted to a non-fluorescent product.
   2.28 Method of Method II, or Methods 2.1-2.13, wherein the detection element comprises fluorescence energy transfer pairs consisting of donor and acceptor molecules.
   2.29 Method of Method II, or any of Methods 2.1-2.13 or 2.28, wherein a flurophore-fused oligonucleotide is the detection element.
   2.30 Method of Method II or Method 2.29, wherein detecting the signal involves exciting the fluorophore with polarized light, measuring emission, and the RNA product will show emission in a parallel channel.
   2.31 Method of Method II, or any of Methods 2.1-2.30, wherein the test compound is FMN or an analogue thereof.
   2.32 Method of Method II, or any of Methods 2.1-2.31, wherein a compound is being tested against any of the disclosed riboswitches.
   The disclosed riboswitches are those herein provided in this specification.
   2.33 Method of Method II, or any of Methods 2.1-2.32, wherein the method may be performed in a high throughput manner.
   2.34 Method of Method II, or any of Methods 2.1-2.33, wherein the step of incubating the DNA sequence includes providing an appropriate concentration of magnesium to modulate the speed of the riboswitch-mediated transcription reactions.
   2.35 Method of Method II, or any of Methods 2.1-2.34, wherein the step of incubating the DNA sequence includes adjusting a pH in order to modulate riboswitch-mediated transcription reactions.
   2.36 Method of Method II, or any of Methods 2.1-2.35, wherein the step of incubating the DNA sequence is optimized by a preceding step of varying a concentration of the DNA sequence among a plurality of parallel in vitro transcription reactions.
   2.37 Method of Method II, or any of Methods 2.1-2.36, wherein the incubation and capturing steps are carried in a pH range from 6-10.
2.38 Method of Method II, or any of Methods 2.1-2.37, wherein the test candidate is an anti-bacterial drug candidate.
   2.39 Method of Method II, or any of Methods 2.1-2.38, wherein the test candidate is a gene expression regulator.
   2.40 Method of Method II, or any of Methods 2.1-2.39, wherein the test candidate is a gene switch regulator.
   2.41 Method of Method II, or any of Methods 2.1-2.40,wherein the incubation and capture steps are conducted in a pH depending on the type of RNA polymerase utilized.
   2.42 Method of Method H or any of Methods 2.1-2.41, wherein the riboswitch sequence is a naturally occurring riboswitch sequence.
   2.43 Method of Method II, or any of Methods 2.1-2.41, wherein the riboswitch sequence is an engineered riboswitch sequence
   2.44 Method of Method II, or any of methods 2.1-2.41, wherein the ribsowitch aptamer domain is an engineered aptamer domain and the riboswitch expression platform domain is an engineered platform domain, which controls transcription of a coding region encoding a signaling sequence.

Thus, for example, the invention provides
1. A method for measuring the effect of a test compound on induction of riboswitch-mediated transcription termination, comprising:
   providing a DNA sequence having a promoter operably linked to a riboswitch aptamer domain, and a riboswitch expression platform, which controls transcription of a coding region encoding a signaling sequence;
   incubating, in the presence or absence of a test compound, the DNA sequence with an RNA polymerase and a plurality of ribonucleotides, e.g., comprising labeled ribonucleotides, to express an RNA product;
   capturing the RNA product having a signaling sequence, using a capture element which specifically recognizes at least a portion of the signaling sequence; wherein the capture element is bound to a substrate;
   removing uncaptured products; and
   detecting the captured RNA product (e.g., by detecting labeled captured RNA product or contacting the RNA product with a labeled detection element which binds to the captured RNA product), wherein a decrease or absence of captured RNA product in the presence of a test compound relative to the signal in the absence of a test compound indicates ligand-induced riboswitch-mediated transcription termination.
2. The method of embodiment 1, wherein the signaling sequence is a defined nucleotide sequence and the capture element is an oligonucleotide complementary to the signaling sequence.
3. The method of embodiment 2, wherein the signaling sequence is a polyA sequence and the capture element is a deoxythymidylate oligonucleotide.
4. The method of any of embodiments 1-3, wherein at least one of the plurality of ribonucleotides is radiolabeled.
5. The method of any of embodiments 1-4, wherein at least one of the plurality of ribonucleotides is labeled by having a radiolabeled ribonucleoside triphosphate.
6. The method of embodiments 4 or 5, wherein the step of detecting the signal of the signaling sequence includes measuring radioactivity, and a decrease in the signal or an absence of the signal indicates a ligand-induced riboswitch-mediated transcription termination.
7. The method of any of embodiments 1-3, wherein at least one of the plurality of ribonucleotides is fluorescently labeled.
8. The method of embodiment 7, wherein the step of detecting the signal of the signaling sequence includes measuring fluorescence intensity, wherein a decrease in the signal or an absence of the signal indicates a ligand-induced riboswitch-mediated transcription termination.
9. The method of any of embodiments 1-8, wherein the step of capturing includes providing the capture element which is an oligonucleotide complementary to the signaling sequence and the step includes hybridizing the RNA product to the oligonucleotide by complementary base pairing.
10. The method of any of embodiments 1 or 4-8, wherein the step of capturing the RNA product with a capture element utilizes affinity binding.
11. The method of embodiment 10, wherein the capture element is an antibody.
12. The method of any of embodiments 1-11, wherein the capture element is biotinylated.
13. The method of embodiment 12, wherein the capture element is immobilized by binding to a streptavidin-coated plate.
14. The method of embodiment 12, wherein the capture element is immobilized using a streptavidin-coated Flash plate that includes an impregnated scintillant.
15. The method of embodiment 12, wherein the capture element is immobilized using a streptavidin-coated bead.
16. The method of embodiment 12, wherein the capture element is immobilized by using a
   a. streptavidin-coated SPA bead.
17. The method of any of the preceding embodiments, wherein the test compound is FMN or an analogue thereof.
18. The method of any of the preceding embodiments, wherein a compound is being tested against any of the disclosed riboswitches.
19. The method of any of the preceding embodiments, wherein the method may be performed in a high throughput manner.
20. The method of any of the preceding embodiments, wherein the step of incubating the DNA sequence includes providing an appropriate concentration of magnesium to modulate the speed of the riboswitch-mediated transcription reactions.
21. The method of any of the preceding embodiments, wherein the step of incubating the DNA sequence includes adjusting a pH in order to modulate riboswitch-mediated transcription reactions.
22. The method of any of the preceding embodiments, wherein the incubation and capture steps are carried out in a pH range from 6-10.
23. The method of any of the preceding embodiments, wherein the step of incubating the DNA sequence is optimized by a preceding step of varying a concentration of the DNA sequence among a plurality of parallel in vitro transcription reactions.
24. The method of any of the preceding embodiments, wherein the test candidate is an anti-bacterial drug candidate.
25. The method of any of the preceding embodiments, wherein the test candidate is a gene expression regulator.
26. The method of any of the preceding embodiments, wherein the test candidate is a gene switch regulator.
27. The method of any of the preceding embodiments, wherein the incubation step is conducted in a pH range depending on the type of RNA polymerase utilized.
28. The method of any of the preceding embodiments, wherein the riboswitch sequence is a naturally occurring riboswitch sequence.
29. The method of any of the preceding embodiments, wherein the riboswitch sequence is an engineered riboswitch sequence.
30. The method of any of the preceding embodiments, wherein the riboswitch aptamer domain is an engineered aptamer domain and the riboswitch expression platform domain is an engineered expression platform domain, which controls transcription of a coding region encoding a signaling sequence.
31. A method for measuring the effect of a test compound on induction of riboswitch-mediated transcription termination, comprising:
   providing a DNA sequence having a promoter operably linked to a riboswitch aptamer domain, and a riboswitch expression platform domain, which control transcription of a coding region encoding a signaling sequence;
      a. incubating, in the presence or absence of a test compound, the DNA sequence with an RNA polymerase and a plurality of ribonucleotides, to express an RNA product;
         capturing the RNA product having a signaling sequence, using a capture element which specifically recognizes at least a portion of the signaling sequence, wherein the capture element is bound to a substrate;
      b. attaching a detection element to the captured RNA product; wherein the detection element specifically recognizes at least a portion of the non-captured portion of the RNA product;
      c. removing uncaptured products; and
      d. detecting the signal generated by the detection element, wherein a decrease or absence of signal of the signaling sequence, in the presence of a test compound relative to the signal in the absence of a test compound indicates ligand-induced riboswitch-mediated transcription termination.
32. The method of embodiment 31, wherein the signaling sequence is a defined nucleotide sequence and the capture element is a complementary oligonucleotide.
33. The method of embodiment 32, wherein the signaling sequence is a polyA sequence and the capture element is a deoxythymidylate oligonucleotide.
34. The method of embodiments 31-33 wherein the step of hybridizing the RNA product to the capture element relies on complementary base pairing.
35. The method of embodiment 31, wherein the step of attaching the capture element to the RNA product utilizes affinity binding.
36. The method of embodiment 35, wherein the capture element is an antibody.
37. The method of embodiments 31-36, wherein the step of attaching the detection element to the captured RNA product utilizes affinity binding.
38. The method of embodiments 31-36, wherein the detection element is an oligonucleotide complementary to a portion of the non-captured portion of the RNA product.
39. The method of embodiment 38, wherein the step of attaching the detection element to the captured RNA product relies on complementary base pairing.
40. The method of any of embodiments 31-39, wherein the capture element is biotinylated.
41. The method of embodiment 40, wherein the capture element is immobilized by binding to a streptavidin-coated plate.
42. The method of embodiment 40, wherein the capture element is immobilized using a streptavidin-coated Flash plate that includes an impregnated scintillant.
43. The method of embodiment 40, wherein the capture element is immobilized using a streptavidin-coated bead.
44. The method of embodiment 40, wherein the capture element is immobilized by using a
   a. streptavidin-coated SPA bead.
45. The method of any of embodiments 31-44, wherein the detection element is labeled.
46. The method of embodiment 45, wherein the detection element is radiolabeled.
47. The method of embodiment 46, wherein the step of detecting the signal of the signaling sequence includes measuring radioactivity, wherein a decrease in the signal or an absence of the signal indicates a ligand-induced riboswitch-mediated transcription termination.
48. The method of any of embodiments 45, wherein the detection element is fluorescently labeled.
49. The method of embodiment 48, wherein the step of detecting the signal of the signaling sequence includes measuring fluorescence intensity, wherein a decrease in the signal or an absence of the signal indicates a ligand-induced riboswitch-mediated transcription termination.
50. The method of any of embodiments 31-44, wherein the detection element is capable of binding to a labeled probe.
51. The method of embodiment 50, wherein the detection element is capable of binding to a radiolabeled probe.
52. The method of embodiment 51, wherein the step of detecting the signal of the signaling sequence includes measuring radioactivity, wherein a decrease in the signal or an absence of the signal indicates a ligand-induced riboswitch-mediated transcription termination.
53. The method of embodiment 50, wherein the detection element is capable of binding to a fluorescently labeled probe.
54. The method of embodiment 53, wherein the step of detecting the signal of the signaling sequence includes measuring fluorescence intensity, wherein a decrease in the signal or an absence of the signal indicates a ligand-induced riboswitch-mediated transcription termination.
55. The method of any of embodiments 31-44, wherein the detection element is biotinylated.
56. The method of embodiment 55, wherein the detecting the signal utilizes streptavidin-conjugated horseradish peroxide (HRP).
57. The method of any of embodiments 55-56, wherein the detecting the signal utilizes a horseradish peroxidase (HRP) substrate.
58. The method of any embodiments 55-57, wherein the HRP substrate is a non-fluorescent HRP substrate that can be converted to a fluorescent product.
59. The method of any embodiments 55-57, wherein the HRP substrate is a non-fluorescent HRP substrate that may be converted to a non-fluorescent product.
60. The method of any of embodiments 31-44, wherein the detection element comprises fluorescence energy transfer pairs consisting of donor and acceptor molecules.
61. The method of any of embodiments 31-44, wherein a flurophore-fused oligonucleotide is the detection element.
62. The method of embodiment 61,wherein detecting the signal involves exciting the fluorophore with polarized light, measuring emission, and the RNA product will show emission in a parallel channel.
63. The method of any of embodiments 31-62, wherein the test compound is FMN or an analogue thereof.
64. The method of any of embodiments 31-63, wherein a compound is being tested against any of the riboswitches disclosed.
65. The method of any of embodiments 31-64, wherein the method may be performed in a high throughput manner.
66. The method of any of embodiments 31-65, wherein the step of incubating the DNA sequence includes providing an appropriate concentration of magnesium to modulate the speed of the riboswitch-mediated transcription reactions.
67. The method of any of embodiments 31-66, wherein the step of incubating the DNA sequence includes adjusting a pH in order to modulate riboswitch-mediated transcription reactions.
68. The method of any of embodiments 31-67, wherein the step of incubating the DNA sequence is optimized by a preceding step of varying a concentration of the DNA sequence among a plurality of parallel in vitro transcription reactions.
69. The method of any of embodiments 31-68,wherein the incubation and capturing steps are carried in a pH range from 6-10.
70. The method of any of embodiments 31-69, wherein the test candidate is an anti-bacterial drug candidate.
71. The method of any of embodiments 31-70 wherein the test candidate is a gene expression regulator.
72. The method of any of embodiments 31-71, wherein the test candidate is a gene switch regulator.
73. The method of any of embodiments 31-72, wherein the incubation step is conducted in a pH depending on the type of RNA polymerase utilized.
74. The method of any of embodiments 31-73, wherein the riboswitch sequence is a naturally occurring riboswitch sequence.
75. The method of any of embodiments 31-73, wherein the riboswitch sequence is an engineered riboswitch sequence.
76. The method of any of embodiments 31-73, wherein the riboswitch aptamer domain is an engineered aptamer domain and the riboswitch expression platform is an engineered platform domain, which controls transcription of a coding region encoding the signaling sequence.
77. A screening assay for testing for anti-bacterial, anti-viral and anti-fungal drug candidates, comprising the method of embodiment 1.
78. A screening assay for testing for anti-bacterial, anti-viral and anti-fungal candidates, comprising the method of embodiment 31.
79. A screening assay kit comprising
   a. a DNA sequence having a promoter operably linked to a riboswitch aptamer domain, and a riboswitch expression platform, which controls transcription of a coding region encoding a signaling sequence;
   b. RNA polymerase and a plurality of ribonucleotides;
   c. a capture element bound to a substrate, which specifically recognizes at least a portion of a signaling sequence of an RNA transcript corresponding to the DNA sequence;
   d. and optionally instructions for use.
80. A screening assay kit providing elements in accordance with any of the foregoing methods.

Drug candidates capable of binding to a riboswitch may be screened in a high throughput assay. A decrease in signal of the signaling sequence may be indicative of a drug candidate's ability to bind productively to a riboswitch and thereby show that the drug candidate is a good inhibitor of bacterial or other pathogenic transcription. If the riboswitch is bound by a drug candidate, lesser amounts of a full-length RNA products will be produced; instead, for example, a truncated RNA product lacking the polyA sequence would be produced.

In one example, a screening assay for testing compounds for anti-bacterial, anti-viral and anti-fungal candidates, comprises the method of Methods I or II or both. Riboswitches are structured RNA elements found in 5′ untranslated regions of the mRNA of certain bacteria, fungi, or plants that regulate the expression of the RNA in which they reside. Riboswitches form a structured receptor that selectively associates with a specific metabolite, and in so doing causing alteration in the structure of the adjoining mRNA in a manner that affects the expression of the operon or open reading frame. In many cases, ligand binding to a riboswitch receptor induces the formation of a terminator hairpin that prevents transcription of the mRNA prior to synthesis of the adjoining operon or open reading frame. In other cases, ligand binding to a riboswitch receptor induces disruption of a terminator hairpin, thereby increasing the expression of the adjoining operon or open reading frame.

In one example, the disclosed methods and the assays may be used for testing compounds and quantitatively measuring the extent to which any ligand among them may induce riboswitch-mediated transcription termination. In one example, a DNA sequence, an RNA polymerase, a suitable buffer for transcription, and ribonucleoside triphosphates (rNTPs) are incubated in the presence of a test compound. The DNA sequence is engineered to include a promoter for initiating transcription, a sequence that encode for a specific riboswitch region, a sequence that encompasses the putative terminator and antiterminator elements, and a 3′-end sequence consisting of multiple adjacent adenylates (polyA). Because it resides downstream of the intrinsic terminator sequence that is under the control of the riboswitch, the polyA sequence will only be expressed when riboswitch-mediated termination does not occur. Subsequent to the transcription reaction, a solid substrate that is coated with immobilized oligodeoxythymidylates may selectively capture polyA sequence-containing full-length transcripts but not truncated transcripts. Quantitation of the amounts of polyA sequence-containing full-length transcripts captured on the substrate under various conditions is accomplished through the inclusion of [α-³³P]-labeled adenosine triphosphate (ATP) in the transcription reaction mixture, which leads to the incorporation of a radiolabel in all transcripts. Thus, in one example, the methods and compositions disclosed provide a procedure to rapidly and easily distinguish between the full-length and truncated transcripts.

In one example, a plate-based assay has been constructed to measure the extent to which a ligand may induce transcription termination by targeting its cognate riboswitch. The example given is the targeting of FMN-responsive riboswitches. In Gram-positive bacteria, FMN-responsive riboswitches control the expression of the mRNA in which they reside through a mechanism that induces premature transcription termination in the presence of an active ligand.

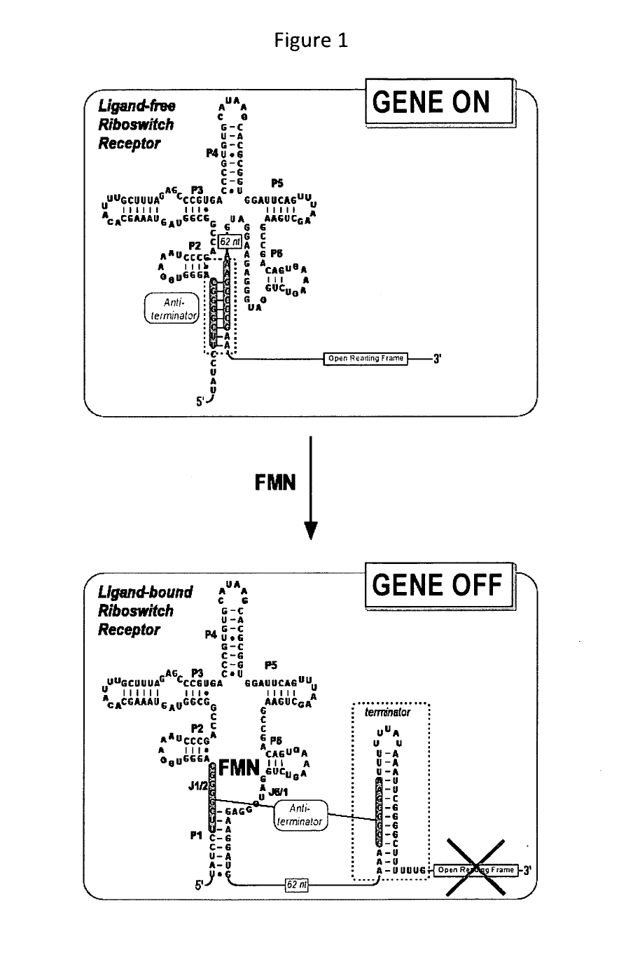

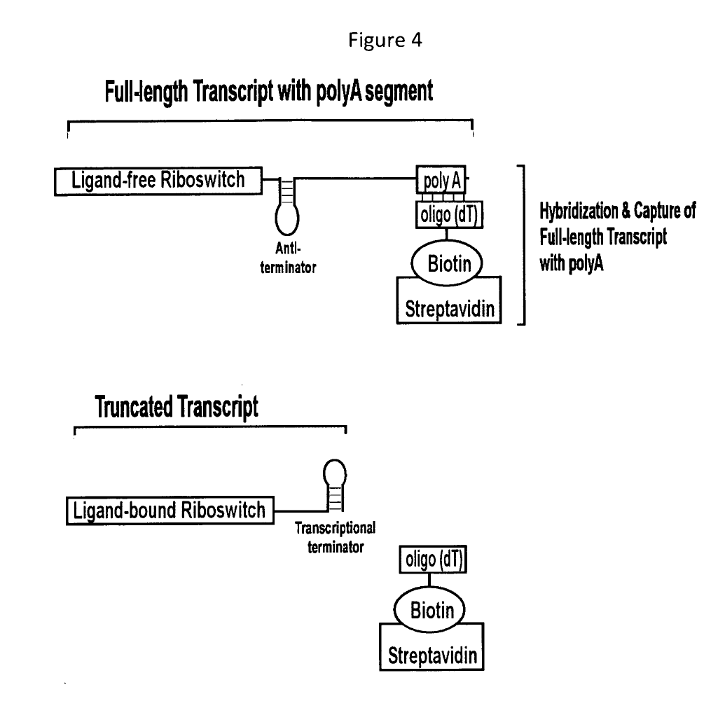

This is accomplished by the ability of the FMN riboswitch to assume one of two alternate conformations of that are dictated by the level of an active ligand. Extensive comparative genomics studies (Gelfand, M. S., Mironov, A. A., Jomnatas, J., Kozlov, Y. I., and Perumov, D. A., 1999 Trends Genet. 15, 439-442, Vitreschak, A. G., Rodionov, D. A., Mironov, A. A., and Gelfand, M. S., 2002 Nucleic Acids Res. 30, 3141-3151) and X-ray crystal structural determination (Serganov, A., Huang, L., and Patel, D., 2009 Nature 458, 233-237) reveal that in the presence of a saturating concentration of FMN, FMN riboswitches fold into a unique structure consisting of a conserved and primarily non base paired core from which five hairpins (paired helical elements) radiate; these are designated P1, P2, P3, P4, and P5 (FIG. 1). The formation of this structure via ligand binding enables the nascent RNA immediately downstream of the riboswitch to form a terminator hairpin (a stable hairpin followed by a stretch of U residues), thus inducing transcription termination. At subsaturating ligand concentration, however, an alternate structure dominates in which nucleotides in the J1/2 region stably form a hairpin structure together with the 5′ proximal nucleotides corresponding to the first half of a terminator hairpin (FIG. 1). This hairpin, termed an antiterminator, prevents the formation of a terminator hairpin, thereby allowing transcription of the entire mRNA. The ribDEAHT has an 5′-untranslated regulatory leader region of approximately 300 base pairs. The conserved regulatory element referred to as the FMN riboswitch is responsible for transcription attenuation of the operon. At lower FMN concentrations, for example, nascent mRNA transcript forms the anti-terminator stem and transcription of the entire mRNA proceeds (FIG. 1). In contrast, at higher FMN concentrations, the FMN riboswitch forms a tight complex with FMN that prevents anti-terminator formation. This allows formation of the terminator stem, resulting in production of truncated mRNA. Multiple methods for detection of the amounts of full-length transcripts are allowable. In one example, a DNA sequence is engineered to include a promoter operably linked to the FMN riboswitch aptamer domain, and a riboswitch expression platform domain, which controls transcription of a coding region encoding an oligonucleotide comprising multiple adjacent adenylates at the 3′-end of the transcript, downstream of the riboswitch receptor and a terminator hairpin. This DNA sequence is incubated, in the presence and absence of a test compound, with an RNA polymerase and a plurality of ribonucleoside triphosphates, wherein one of the plurality of ribonucleoside triphosphates is radiolabeled. After the full-length transcripts having a polyA sequence are generated by an in vitro transcription reaction, biotinylated oligo deoxythymidylates (oligo (dT)) are hybridized to the polyA sequence via complementary base-pairings. The resulting hybridized RNA products are then captured by binding to a streptavidin-coated substrate for a radiometric measurement (FIG. 4). By contrast, the truncated transcripts lacking a polyA sequence are not capable of hybridizing to oligo(dT) and are not captured. All uncaptured products are removed before a radiometric measurement is performed.

There are published methods to assess the effects of a potential ligand on riboswitch functions. Most frequently, full-length and truncated transcripts have been separated and quantitated by polyacrylamide gel electrophoresis (PAGE). This method suffers from the labor intensive process of setting up, running, and then analyzing the gel images. Such a method is space- and time-consuming and thus not amenable to screening hundreds or thousands of compounds in a high throughput manner. An HTS-compatible assay to assess activity of the glmS riboswitch has also been described. Since this assay relies for detection on ligand-induced ribozyme activity, it is not applicable to other known naturally occurring riboswitches. Moreover, this previously disclosed method does not allow whether or not a ligand induces transcription termination. It has been well documented that different classes of riboswitches in some bacterial pathogens regulate the expression of a gene or a cluster of genes essential for cell growth, survival, or virulence. It has been proposed that small molecules designed to bind to particular riboswitches should be able to modulate riboswitch-mediated processes with deleterious effects on the pathogens (i.e., cell growth inhibition, cell cycle arrest, and drug-induced apoptosis).

### EXAMPLES

### Example 1

### Cloning of Riboswitch Domains

### Example 1A

### FMN Responsive Riboswitch

The FMN riboswitch within the leader sequence of the B. subtilis ribDEAHT operon was amplified by PCR from B. subtilis strain 168 (Bacillus Genetic Stock Center—designation 1A1). PCR of a B. subtilis genomic preparation is performed using Platinum® Taq DNA Polymerase High Fidelity from Invitrogen (catalog #11304-011) and the sense PCR and the antisense polyA PCR primers (SEQ ID: NO 1 and 2, or SEQ ID: NO 1 and 3, respectively, as shown in table below). PCR using Taq polymerase resulted in a single overhanging deoxyadenylate residue at each 3′-end. These overhangs allow the ligation of DNA into the pCR®2.1-TOPO® vector using a TOPO TA Cloning® kit (Invitrogen catalog #K4500-01). Following transformation, colonies that contained disrupted β-galactosidase are picked and grown overnight. Correct insertion and orientation of PCR product are verified by restriction analysis and sequencing. PCR of the resulting clone is used to generate DNA sequences for use in in vitro transcription termination assays. As a negative control, the sense PCR primer (SEQ ID: NO1) is used with the antisense polyT PCR primer (SEQ ID: NO4).

### Example 1B

### TPP-Responsive Riboswitch

The TPP riboswitch within the leader sequence of the B. subtilis tenA operon is amplified by PCR from B. subtilis strain 168 (Bacillus Genetic Stock Center—designation 1A1). PCR of a B. subtilis genomic preparation was performed using Platinum® Taq DNA Polymerase High Fidelity from Invitrogen (catalog #11304-011) and the sense PCR and the antisense polyA PCR primers (SEQ ID: NO 6 and 7, respectively). PCR using Taq polymerase resulted in a single overhanging deoxyadenylate residue at each 3′-end. These overhangs allow the ligation of DNA into the pCR®2.1-TOPO® vector using a TOPO TA Cloning® kit (Invitrogen catalog #K4500-01). Following transformation, colonies that contained disrupted β-galactosidase were picked and grown overnight. Correct insertion and orientation of PCR product were verified by restriction analysis and sequencing. PCR of the resulting clone was used to generate DNA sequences for use in in vitro transcription termination assays.

### Example 1C

### SAM-Responsive Riboswitch

The SAM riboswitch within the leader sequence of B. subtilis yicI operon was amplified by PCR from B. subtilis strain 168 (Bacillus Genetic Stock Center—designation 1A1). PCR of a B. subtilis genomic preparation was performed using Platinum® Taq DNA Polymerase High Fidelity from Invitrogen (catalog #11304-011) and the sense PCR and the antisense polyA PCR primers (SEQ ID NO: 8 and 9, respectively). Resulting PCR product is used as template in in vitro transcription assays.

Primer sequences are shown in the table below. Sense PCR primers and antisense polyA primers are shown below for exemplification as an example. Similarly, a biotinylated oligo (dT) shown in the table below is shown here as an example. A person of ordinary skill in the art may easily modify the length and composition of any oligonucleotides.

| Oligonucleotide | Sequence (5′ to 3′) | Length |
|---|---|---|
| Sense PCR | CTGAATTCTTTCGGATCGAAGGGTG | 25mer |
| primer | | |
| (SEQ ID | | |
| NO: 1) | | |
| Antisense | TTTTTTTTTTTTTTTTTTTTTTTACTCTTCCATTTGTTTCCC | 42mer |
| polyA PCR | | |
| primer | | |
| (SEQ ID | | |
| NO: 2) | | |
| Antisense | TTTTTTTTTTTTTTTTTTTTTTTTTTTTTTTTTTTTTTTACTCTTCCATTTGTTTCC | 58mer |
| polyA PCR | C | |
| primer | | |
| (SEQ ID | | |
| NO: 3) | | |
| Antisense | AAAAAAAAAAAAAAAAAAAAAATACTCTTCCATTTGTTTCCC | 42mer |
| polyT PCR | | |
| primer | | |
| (SEQ ID | | |
| NO: 4) | | |
| Oligo (dT) | biotinylated-TTTTTTTTTTTTTTTTTTTTTTTTT | 25mer |
| (SEQ ID | | |
| NO: 5) | | |
| Sense PCR | CTGAATTCGGGTACGTAACGGATCT | 25 mer |
| primer | | |
| (SeqID | | |
| NO: 6) | | |
| Antisense | TTTTTTTTTTTTTTTTTTTTTTTACTGCGGCATTCTTCTG | 40 mer |
| polyA PCR | | |
| primer | | |
| (SEQ ID | | |
| NO: 7) | | |
| Sense PCR | CTGAATTCCTTTAAACGGTTCGGCACACG | 29mer |
| primer | | |
| (SeqID | | |
| NO: 8) | | |
| Antisense | TTTTTTTTTTTTTTTTTTTTTTTCGTGCTGTGACATATAGCTG | 43 mer |
| polyA PCR | | |
| primer | | |
| (SEQ ID | | |
| NO: 9) | | |

### Example 2

### PolyA-Sequence Containing Transcription Product of FMN Riboswitch-Mediated Transcription Reactions are Captured and Retained for Detection

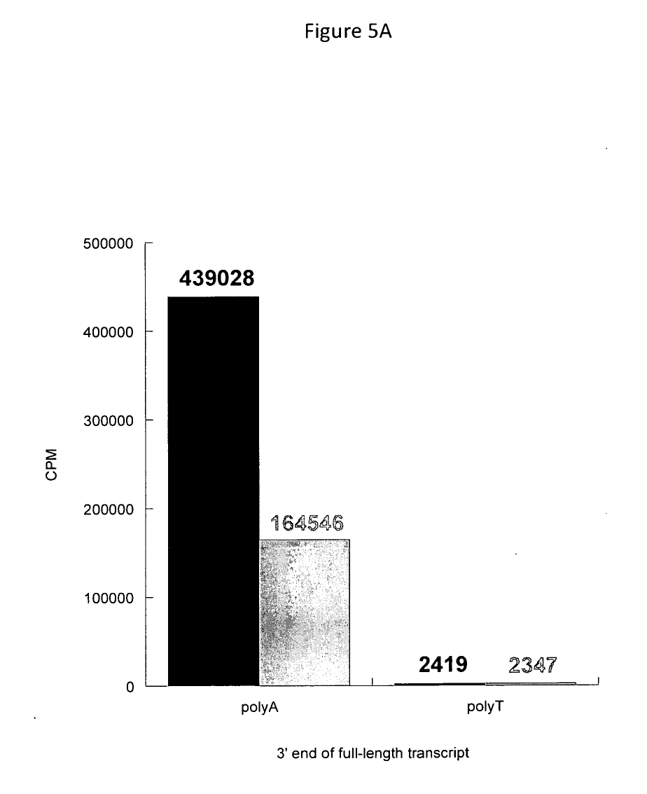

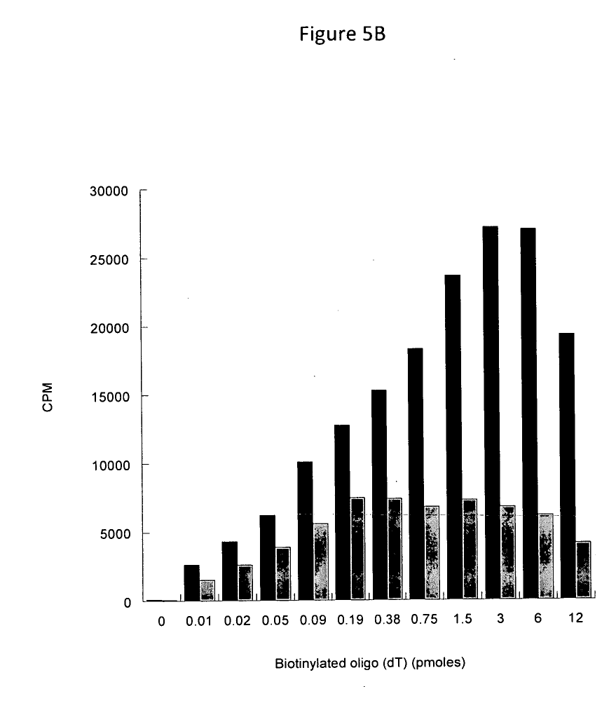

To demonstrate that an RNA transcript with a polyA sequence is able to bind to biotinylated oligo (dT), allowing capture and retention of the RNA transcript within the well, two DNA sequences are compared. These two DNA sequences encode the same full-length transcripts but differ only at the 3′ end. One DNA sequence is engineered to generate a polyA sequence consisting of 23 adenylate nucleotides at the 3′-end, and the other DNA sequence is designed to generate a polyT sequence comprising 22 thymidylate nucleotides. As shown in FIG. 5A, the two DNA sequences are used in in vitro transcription reactions in the absence or presence of 100 μM FMN.

Synthetic DNA primers and a biotinylated oligo (dT) may be obtained from the Keck Foundation Biotechnology Resource Center at Yale University. Flavin mononucleotide (FMN) may be purchased from Fluka BioChemika (catalog #83810). [α-³³P] labeled Adenosine 5′ triphosphate was acquired from American Radiolabeled Chemicals, Inc. (catalog #ARP0131). In vitro plate-based transcription assay: In vitro transcription reactions are carried out at 25° C. in a total reaction volume of 50 μL. Reactions are performed in Reacti-Bind™ Streptavidin High Binding Capacity Coated 96-Well Plates (Pierce, catalog #15502) in assay buffer at a final concentration of 20 mM HEPES at pH 8.0, 60 mM KCl, 0.1 mM EDTA, and 0.005% BSA. To prepare plates, wells are washed three times with successive addition and aspiration of wash buffer containing 25 mM Tris-HCl at pH 7.4 and 150 mM NaCl. Each well is then given assay buffer, followed by addition of FMN into control wells or DMSO into test wells. Enzyme mixture containing sigma-saturated E. coli polymerase (Epicentre Biotechnologies, catolog #S90250) was then added to all wells. The reaction was initiated by the addition of a start mix containing ribonucleotides (Promega, Catalog #P1300), biotinylated oligo (dT)₂₅, magnesium, [α-³³P]-labeled ATP and DNA sequence. The final concentrations of FMN, RNA polymerase, ribonucleotides (rNTPs), biotinylated oligo (dT), Mg²⁺, [α-³³P]-labeled ATP, and DNA sequence in the reaction mixtures all varied according to the particular experiment and are shown in the figure legends and examples. The amount of DNA sequence used in transcription reactions is reported as units of μL of PCR reaction product. Concentrations of rNTPs are listed as the concentration of each of four ribonucleotides (rATP, rCTP, rGTP, and rUTP), respectively. After reaction is initiated, plates are sealed and reactions are incubated for 3 hours, unless otherwise stated. An addition of 13 μL of stop solution containing 2.4 M NaCl and 0.24 M EDTA quenched the reaction. The quenched reaction mixture is incubated for another 60-90 minutes at 25° C. The wells are then washed two times with a high salt wash buffer containing 20 mM Tris-HCl, pH 8.0 and 500 mM NaCl. During the wash step, any remaining unincorporated radiolabeled ATP, test compounds, and truncated transcripts are removed. 50 μl of Microscint™PS (PerkinElmer, catalog #6013631) are then added to wells and the plate is read in a TopCount®NXT™ microplate scintillation and luminescence counter (PerkinElmer). Other readers and counters may be also be used.

In vitro transcription reactions for this Example are carried out under the following conditions: 12.5 μM of each rNTP, 2.5 mM Mg²⁺, 20 pmoles biotinylated oligo (dT), 1 μCi [α-³³P]-ATP, 1 μl DNA sequence, and 0.25 Unit sigma-rich RNA polymerase. The dark grey bars and the light grey bars in FIG. 5A represent reactions minus and plus FMN, respectively. Quantitatation of the amount of RNA transcript captured on the plate is accomplished by the incorporation of [α-³³P]-labeled nucleotide during in vitro transcription reaction and subsequent radiometric measurement. RNA transcript with a polyT sequence generated less than 1% of the signal generated by the RNA transcript with a polyA sequence (dark bars). In the presence of FMN, a decrease in signal of the polyA sequence is shown. Lesser amounts of full-length RNA transcript having the polyA sequence are expressed, due to FMN binding to a target riboswitch.

During transcription, FMN binds to the FMN riboswitch and induces transcription termination, resulting in the formation of a truncated transcription product lacking a polyA sequence. The truncated product does not interact with the oligo (dT) and is therefore not captured. The truncated product instead is eliminated during the wash steps. The addition of FMN to the transcription reaction with DNA sequence encoding a polyA sequence resulted in a significant decrease in signal (“polyA” dark vs. light grey bar).

In order for a RNA transcript with a polyA sequence to be captured and retained within a streptavidin-coated well, biotinylated oligo (dT) is first hybridized to the RNA transcript having the polyA sequence via base-pairing between the deoxythymidylate and the adenylate and immobilized by binding to streptavidin. In FIG. 5B, oligo (dT) is titrated plus and minus FMN. In vitro transcription reactions are carried out as described above under the following conditions: 12.5 μM each rNTP, 2.5 mM Mg²⁺, 0.125 μCi [α⁻³³P]-ATP, 0.25 μL DNA sequence, 0.25 Unit RNA polymerase, and varying concentrations of biotinylated oligo (dT) as indicated. The dark grey bars and the light grey bars represent reactions minus and plus FMN, respectively. Data in FIG. 5B show oligo(dT) dependence as demonstrated by a gradual increase in signal from background at 0 pmole to a maximum signal at approximately 3 pmoles oligo(dT) (dark grey bars). FMN results in a decrease in signal at each oligo(dT) concentration due to decreased production of full-length transcript with a polyA sequence (light grey vs. dark grey bars).

### Example 2

### Riboswitch-Mediated in vitro Transcription Reactions Dependent on Magnesium and Ribonucleotides

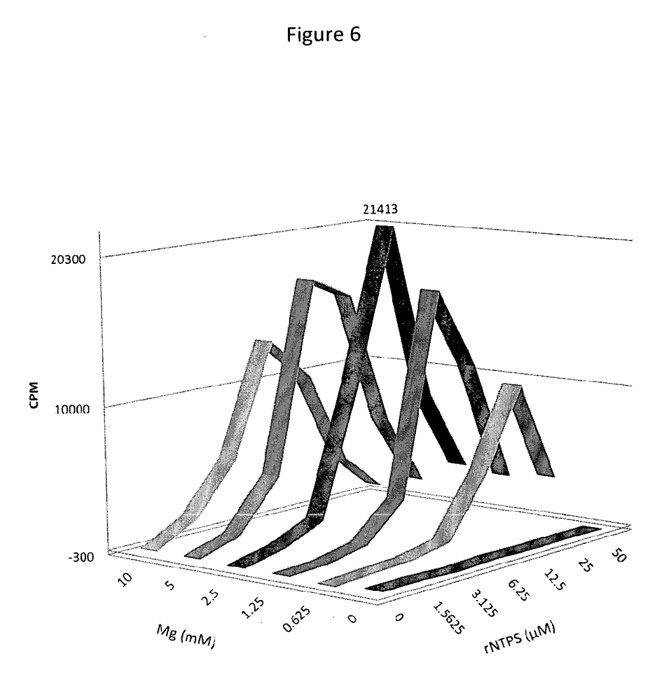

It has been demonstrated that FMN riboswitch-mediated transcription relies on the relative speed of metabolite binding and RNA polymerase reaction (Wickiser, J. K., Winkler, W. C., Breaker, R. R., and Crothers D. M., Molecular Cell 2005, 18, 49-6). When RNA polymerase transcribes too quickly, FMN and FMN analogues do not have sufficient time to bind to the riboswitch receptor to form a complex that would permit the formation of the terminator hairpin. Under these conditions, FMN will have no significant effect on induction of riboswitch-mediated transcription termination regardless of its concentration. Because the concentration of rNTPs or Mg²⁺ or both may influence the speed of a transcription reaction, Mg²⁺ and rNTP are titrated to determine their effect on FMN riboswitch-mediated transcription termination. FIG. 6 shows that rNTPs are titrated at multiple Mg²⁺ concentrations plus and minus 100 μM FMN. The calculated windows, as defined by the difference in radiometric measurements of [³³P]-labeled transcript with and without FMN, are plotted at all Mg²⁺ and rNTPs combinations. In vitro transcription reactions are carried out as described above under the following conditions: 3 pmoles biotinylated oligo (dT), 0.125 μCi [α-³³P]-ATP, 0.25 μL DNA sequence, 0.1 Unit RNA polymerase, varying Mg²⁺, and varying rNTPs as indicated in FIG. 6. Window, as defined by the difference in signal (cpm) with and without FMN, is plotted. RNA polymerase is a nucleotidyl transferase that requires magnesium to polymerize ribonucleotides at the 3′ end of an RNA transcript. The data in FIG. 6 reveals that the polymerase reaction is dependent on both magnesium and rNTPs. The decrease in cpm observed at high concentration of rNTPs is due to a competition of excess unlabeled ATP with the fixed amount of radiolabeled ATP. Under these conditions, fewer RNA transcripts are radiolabeled due to a low specific activity.

### Example 3

### Riboswitch-Mediated in vitro Transcription Reactions are DNA Sequence Dependent and Signal is Proportional to DNA Sequence Concentration

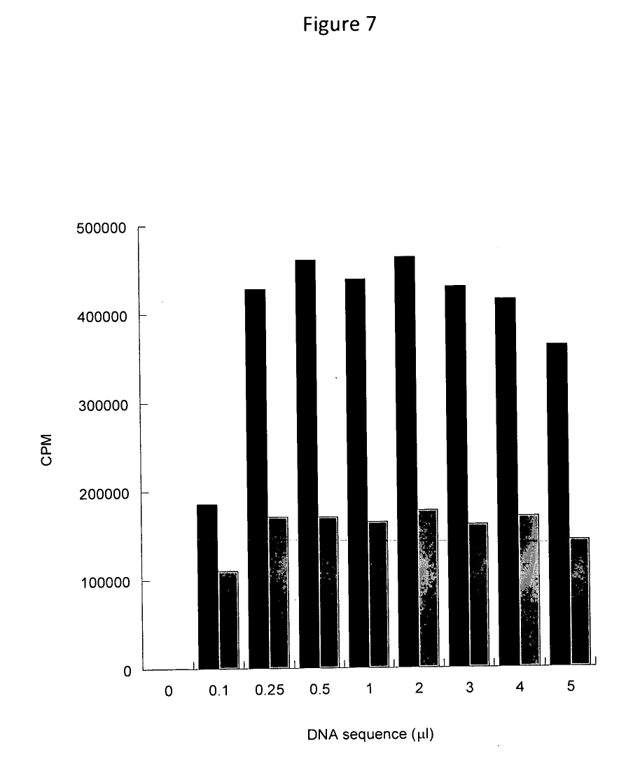

During transcription, RNA polymerase assembles an RNA polynucleotide that is complementary to a DNA sequence. In FIG. 7, DNA sequence is titrated plus (light grey bars) and minus (dark grey bars) 100 μM FMN. In vitro transcription reactions are carried out as described above under the following conditions: 12.5 μM each rNTP, 2.5 mM Mg²⁺, 20 pmoles biotinylated oligo (dT), 1 μCi [α-³³P]-ATP, 0.25 Unit RNA polymerase, and varying DNA sequence as indicated in the figure. FIG. 7 shows that both signal and window are DNA sequence dependent. The signal and window increase with increasing DNA sequence and level off at approximately 0.25 μL DNA sequence. For example, in the presence of a riboswitch-binding compound, such as FMN, a decreased signal is observed, as shown in FIG. 7.

### Example 4

### Riboswitch-Mediated in vitro Transcription Reactions are Enzyme Dependent and Time Dependent

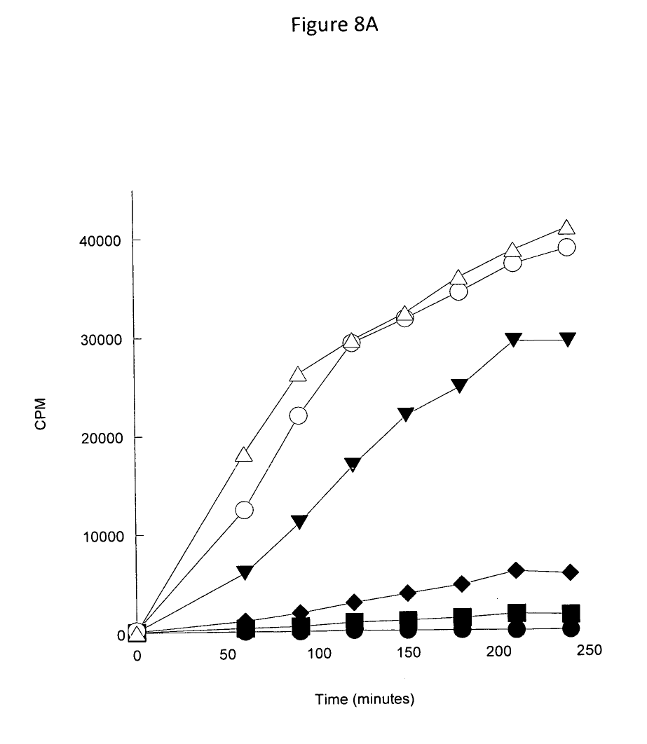

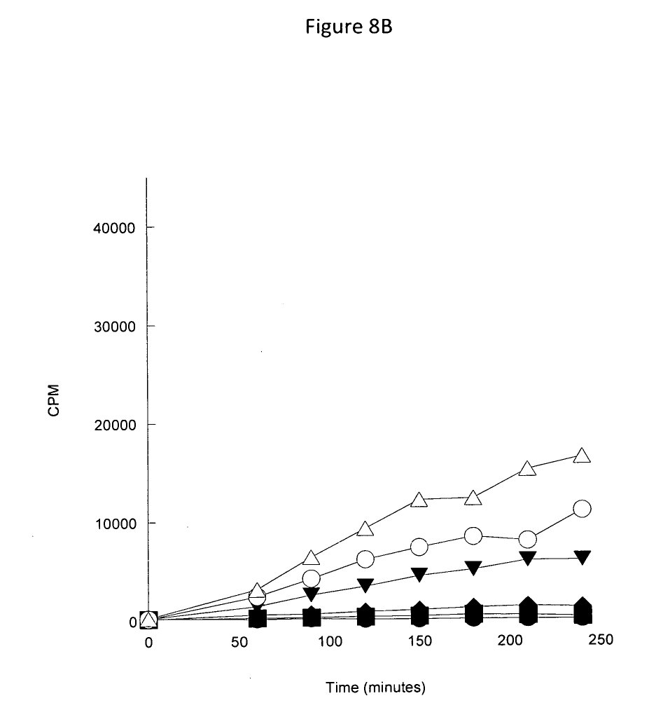

The amount of product formed during an enzymatic reaction increases with time until substrate is consumed, and is proportional to enzyme concentration. To demonstrate this, RNA polymerase is titrated in time course reactions in the absence (FIG. 8A) and presence (FIG. 8B) of 100 μM FMN. In vitro transcription reactions are carried out as described above under the following conditions: 12.5 μM each rNTP, 2.5 mM Mg²⁺, 3 pmoles biotinylated oligo (dT), 0.125 μCi [α-³³P]-ATP, and 0.25 μL DNA sequence.

Stop solution is added to the appropriate wells at each time point. Enzyme concentrations in FIGS. 8A and 8B are labeled as follows: 0.2 Unit (Δ), 0.15 Unit (∘), 0.1 Unit (▾), 0.05 Unit (♦), 0.025 Unit (▪), 0.0125 Unit (). FIG. 8A reveals that signal is time dependent and is proportional to enzyme concentration. The same is true in the presence of FMN (FIG. 8B) although lower signals are observed due to FMN riboswitch-mediated transcription termination. The signal is linear for at least 3.5 hours and the window increases with time.

FIG. 8A shows that as the concentration of the RNA polymerase increases in the absence of FMN, more full-length RNA products having the polyA sequence are produced. Thus, an increase in signal of the polyA sequence is shown. In FIG. 8B, there is still a dependence on the signal of the polyA sequence on the amount of the RNA polymerase, but the signal is reduced in the presence of a riboswitch binding compound such as FMN.

### Example 5

### Riboswitch-Mediated in vitro Transcription Reactions are pH Dependent

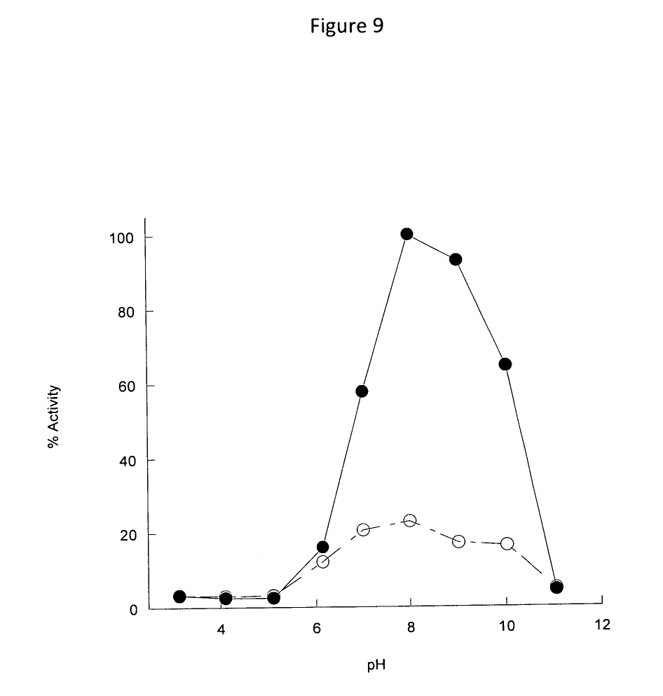

To show pH dependence for riboswitch-mediated transcription using E. coli RNA polymerase, in vitro transcription reactions are performed while varying pH of the reaction buffer from 3 to 11. As shown in FIG. 9, pH is titrated minus () and plus (∘) 100 μM FMN. In vitro transcription reactions are carried out as described above under the following conditions: 12.5 μM each rNTP, 2.5 mM Mg²⁺, 3 pmoles biotinylated oligo (dT), 0.125 μCi [α-³³p]-ATP, 0.1 Unit RNA polymerase, and 0.25 μL DNA sequence. Phosphate buffer is used to obtain the desired pH. The lines in FIG. 9 are drawn to connect symbols. Data in FIG. 9 demonstrate that both the total signal and window are pH dependent. A person of ordinary skill in the art may easily modify the pH conditions. For example, one may use an RNA polymerase obtained from acidophilic bacteria. Alternatively, one may use an RNA polymerase derived from alkalophilic bacteria. Thus, the pH range profile may vary depending on the type of RNA polymerase used.

### Example 6

### Rifampin Inhibits RNA Polymerase Activity

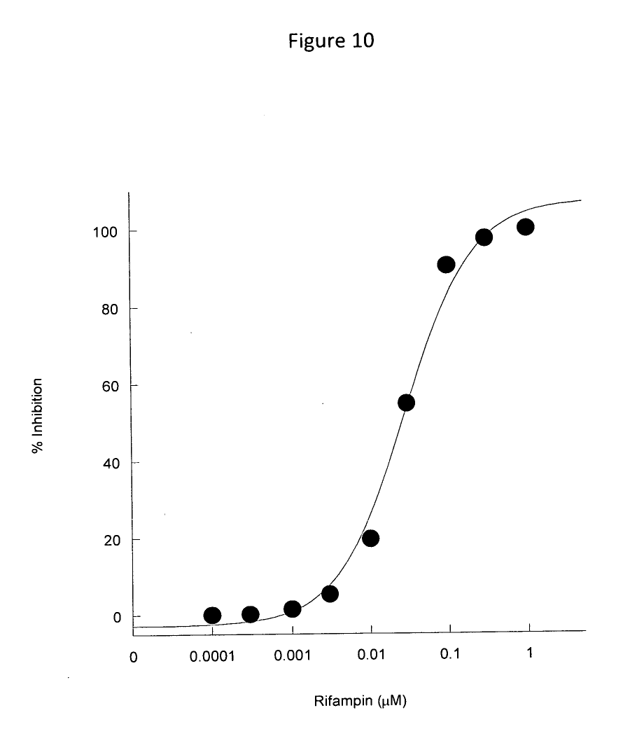

The antibacterial rifampin is an RNA polymerase inhibitor (Liu, J., Feldman, P. A., Lippy, J. S., Bobkova, K., Kurilla, M. G, and Chung, T. D. Y., 2001 Analytical Biochemistry, 289, 239-245, Fujii, K., Saito, H., Tomioka, H., Mae, and T., Hosoe, K., 1995 Antimicrobial Agents and Chemotherapy, 39, 1489-1492, Morris, A. B., Brown, R. B., and Sands, M., 1993 Antimicrobial Agents and Chemotherapy 37, 1-7). Rifampin is titrated to inhibit in vitro transcription reaction and its inhibitory IC₅₀ value is determined (FIG. 10). In vitro transcription reactions are carried out as described above under the following conditions: 12.5 μM each rNTP, 2.5 mM Mg²⁺, 3 pmoles biotinylated oligo (dT), 0.125 μCi [α-³³P]-ATP, 0.1 Unit RNA polymerase, and 0.25 μL DNA sequence.

The IC₅₀ value for inhibition of the in vitro transcription reaction by rifampin is determined from the data shown in FIG. 10. Data are fit with KaleidaGraph software using a three parameter non-linear logistic model. The IC₅₀ value of 8.2 nM from FIG. 10 is in close agreement with IC₅₀ values reported in literature. (Liu, J., Feldman, P. A., Lippy, J. S., Bobkova, K., Kurilla, M. G, and Chung, T. D. Y., 2001 Analytical Biochemistry, 289, 239-245, Fujii, K., Saito, H., Tomioka, H., Mae, and T., Hosoe, K., 1995 Antimicrobial Agents and Chemotherapy, 39, 1489-1492).

### Example 7

### High Throughput Screening

To demonstrate the utility of this assay for identifying compounds that modulate FMN-riboswitch mediated transcription, the assay, in one example, is performed using 96-well density microtiter plates under simulated HTS conditions. In vitro transcription reactions are carried out as described above under the following conditions: 12.5 μM each rNTP, 2.5 mM Mg²⁺, 3 pmoles biotinylated oligo (dT), 0.125 μCi [α-³³P]-ATP, 0.25 μL DNA sequence, and 0.1 Unit RNA polymerase. The protocol includes the following steps: (1) addition of 30 μL of assay buffer (2) addition of 5 μL of DMSO or test compound (3) addition of 5 μL of RNA polymerase (4) addition of 10 μL of start mix followed by sealing of a plate (5) incubation for 3 hours at 25° C. (6) addition of 13 μL of quench/binding solution, followed by sealing of a plate (7) incubation for 90 minutes (8) aspiration of reaction mix from wells, followed by two washes (9) addition of 50 μL of scintillation cocktail, followed by sealing of a plate (10) plate read in a TopCount®NXT™ microplate counter. Other counters may be used.

A DMSO (dimethyl sulfoxide) plate is tested to evaluate the assay quality under the HTS conditions described above. The 96-well DMSO plate consists of negative control wells treated with 0 μM FMN, positive control wells treated with 100 μM FMN, and DMSO treated wells. Signals (cpm) from each well of the 96-well DMSO plate are plotted and include 0 μM FMN added, 100 μM FMN added, and DMSO added. Percent inhibition is calculated based on the window between in vitro transcription reactions treated with and without 100 μM FMN where 100% inhibition is defined as signal observed at 100 μM FMN. The value halfway between the negative and positive controls is labeled as “50% threshold”. The signal-to-background of the assay under current conditions in a 3 hr in vitro transcription reaction is ˜7-fold with a window of ˜19,000 cpm. The performance of this assay in HTS mode is assessed by calculating the Z′ factor from the DMSO plate (Zhang, J., Chung, T. D. Y., and Oldenburg, K., 1999 J of Biomolecular Screening, 4, 67-73). The Z′ factor is a relative measure of whether the separation between the negative (0 μM FMN) and positive (100 μM FMN) populations is statistically significant. The Z′ value calculated in one experiment is 0.57. In general, a value of Z′≧0.5 indicates that the assay is of high quality and suitable for HTS.

In vitro transcription assay is performed under simulated HTS manner to test a compound library plate under the conditions described above. The screening is performed at the final concentration of 10 μM compound. In vitro transcription reactions are carried out as described above under the following conditions: 12.5 μM each rNTP, 2.5 mM Mg²⁺, 3 pmoles biotinylated oligo (dT), 0.125 μCi [α-³³P]-ATP, 0.1 Unit RNA polymerase, and 0.25 μL DNA sequence. In FIG. 10A, the rows and columns of an overhead view of the compound plate are labeled A-H and 1-12, respectively: no RNA polymerase in wells A1-B1 (0×), 0.1 Unit RNA polymerase in wells C1-F1 (1×), 0.2 Unit RNA polymerase in wells G1-H1 (2×), 40 nM FMN in wells A2-D2 (40 nM FMN), 100 μM FMN in wells E2-H2 (100 μM FMN), and test compounds in well A3-H12 (plate 1×). Percent inhibition is calculated based on the differences in signals (cpm) between no FMN and 100 μM FMN. Test compounds that significantly alter the amount of full-length transcript are identified as “hits.” Hit identification is based on statistical analysis of screening data and determination of an appropriate threshold for a particular screening campaign. Compounds that would meet the criteria will then be identified as hits. For example, wells containing test compounds that show greater than 50% inhibition are deemed to be “hits” under current conditions.

### Example 8

### Functional Effects of FMN and FMN Analogues on in vitro Transcription are Evaluated

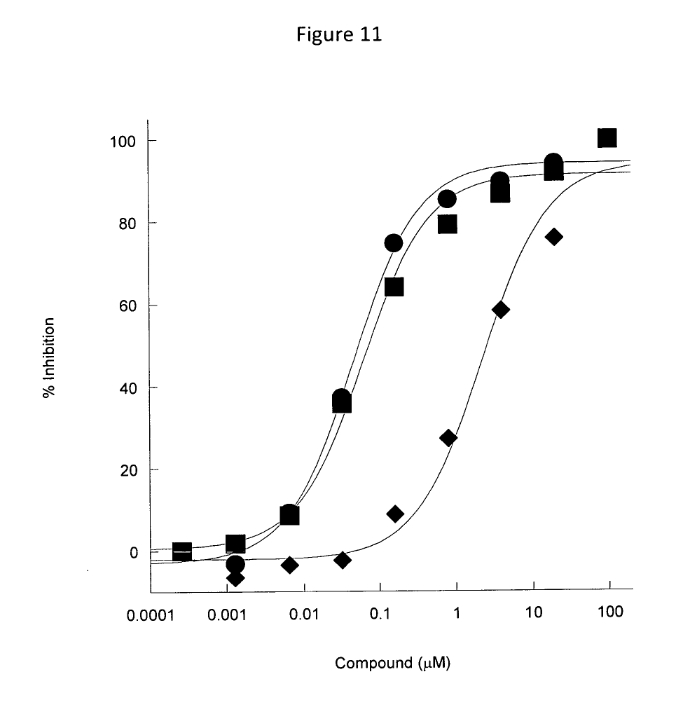

FMN has been shown to modulate FMN-riboswitch mediated transcription in a dose dependent manner. FMN and proprietary FMN analogues are titrated in in vitro transcription reactions to determine IC₅₀ values for each compound. In vitro transcription reactions are carried out as described above under the following conditions: 12.5 μM each rNTP, 2.5 mM Mg²⁺, 3 pmoles biotinylated oligo (dT), 0.125 μCi [α-³³]-ATP, 0.25 μDNA sequence, and 0.1 Unit RNA polymerase. FIG. 11 describes dose response curves of three compounds including FMN (), FMN analogue 1 (▪), and FMN analogue 2 (♦) with IC₅₀ values of 45 nM, 60 nM, and 2200 nM, respectively. This is an example of an induction of in vitro riboswitch-mediated transcription termination by FMN and FMN analogues. Data are fit with KaleidaGraph software using a three parameter non-linear logistic model.

### Example 9

### Assay Using TPP-Responsive Riboswitch as Target

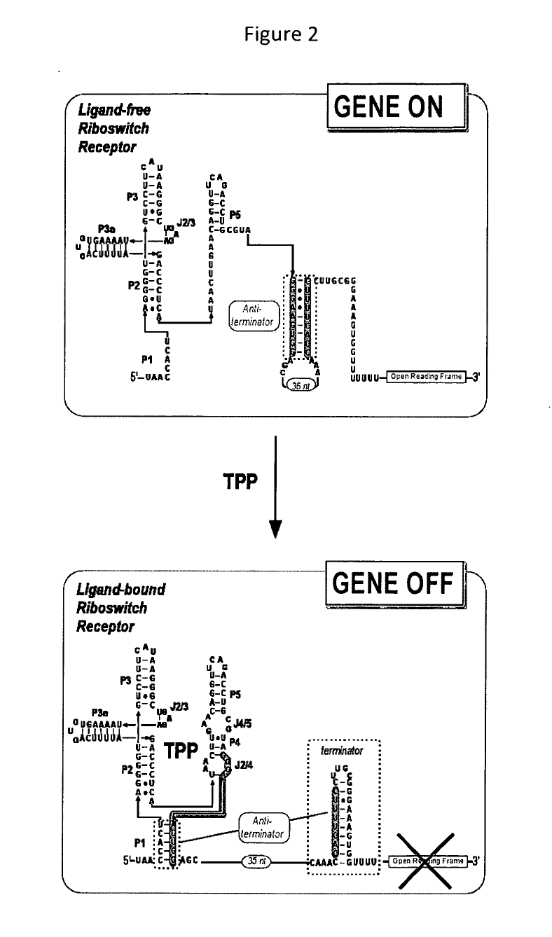

Another example given herein is the targeting of TPP-responsive riboswitches. The regulation of thiamine genes in Gram-positive bacteria has been well characterized. In general, thiamine and TPP downregulate thiamine gene expression (Begley, T. P., Downs, D. M., Ealick, S. E., Mclafferty, F. W., Van Loon, A. P., Taylor, S., Campobasso, N., Chiu, H. J., Kinsland, C., Reddick, J. J., and Xi, J., 1999 Arch. Microbiol, 171, 293-300, Petersen, L. A. and Downs, D. M., 1997, J. Bacteriol. 179, 6887-6893). Similar to FMN-reponsive riboswitches, as described above, TPP-responsive riboswitches in Gram-positive bacteria, control the expression of the mRNA by a mechanism that induces premature transcription termination in the presence of an active ligand. This is accomplished by the ability of the TPP riboswitch to assume one of two alternate conformations that are dictated by the level of an active ligand (Winkler, W., Nahvi, A., and Breaker, R. R. 2002 Nature 419, 953-956, Mironov, A. S., Gusarov, I., Rafikov, R., Lopez, L. E., Shatalin, K., Kreneva, R. A., Perumov, D. A., and Nudler, E. 2002, Cell 111, 747-756). TPP riboswitches comprise one subdomain that recognizes the polar functional group of the 4-amino-5-hydroxymethyl-2-methylpyrimidine (HMP) moiety and another subdomain that coordinates two metal ions and several water molecules to bind the negatively charged pyrophosphate moiety of the ligand (Serganov, A., Polonskaia, A., Phan, A. T., Breaker, R. R. and Patel, D. J. 2006 Nature 441, 1167-1171, Edwards, T. E. and Ferre-D'Amare, A. R. 2006 Structure 14, 1459-1468). The formation of this structure via ligand binding enables the nascent RNA immediately downstream of the riboswitch to form a terminator hairpin (a stable hairpin followed by a stretch of U residues), thus inducing transcription termination (FIG. 2). At saturating concentrations of TPP, for example, TPP riboswitches fold into a unique receptor structure comprised of five helices (P1 through P5) joined by three stretches of primary unpaired nucleotides (J2/3, J2/4 and J4/5). The formation of the base-paired structure (P1) in TPP-bound riboswitches allows a terminator hairpin to form. This results in the induction of transcription termination. Alternatively, if TPP is not present during the synthesis of the 5′UTR and no TPP is bound to the TPP riboswitches, the nucleotides in P1 are freely available to form an antiterminator hairpin (FIG. 2). This hairpin, termed an antiterminator, prevents the formation of a terminator hairpin, thereby allowing transcription of the entire mRNA.

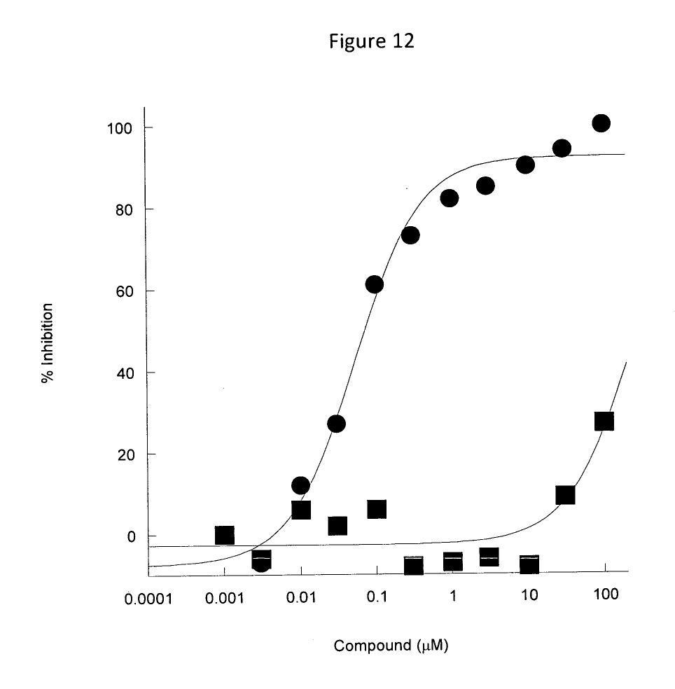

Synthetic DNA primers and a biotinylated oligo (dT) may be obtained from the Keck Foundation Biotechnology Resource Center at Yale University. Thiamine pyrophosphate (TPP, catalog #C8754), Thiamine monophosphate (TMP, catalog #T8637) may be purchased from Sigma. [α-³³P] labeled Adenosine 5′ triphosphate may be acquired from American Radiolabeled Chemicals, Inc. (catalog #ARP0131). RiboGreen® RNA quantitation reagent may be purchased from Invitrogen/Molecular Probes (catalog #R-11491). FIG. 12 shows an assay using TPP and TPP analogs.

### Example 10

### Assay Targeting SAM-Responsive Riboswitches

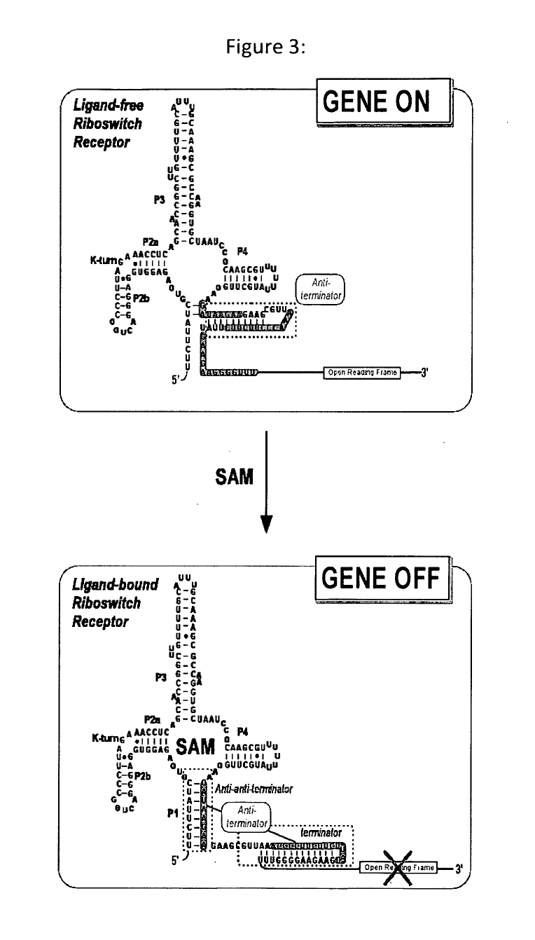

Yet another example given herein is the targeting of SAM-responsive riboswitches. In Gram-positive bacteria, a variety of bacteria species present an evolutionary conserved regulatory leader sequence (S-box leader) that are involved in sulfur metabolism, amino acid metabolism (Cys and Met biosynthesis) and SAM biosynthesis (Grundy, F. J. and Henkin, T. M., 1998 Mol. Microbiol. 30, 737-749, Grundy, F. J. and Henkin, T. M., 2003 Front. Biosci. 8, d20-d31, Epshtein, V., Mironov, A. S., and Nudler, E. 2003 PNAS USA 100, 5052-5056. Winkler, W. C., Nahvi, A., Sudarsan, N., Barrick, J. E., and Breaker R. R. 2003 Nat. Struct. Biol. 10, 701-707). The leader sequence of SAM-responsive riboswitch controlled genes include an intrinsic transcription terminator, competing antiterminator and an S-box that can function as the anti-antiterminator. Analogous to FMN- and TPP-reponsive riboswitches, as described above, SAM-responsive riboswitches control the expression of the mRNA in which they reside through a mechanism that induces premature transcription termination in the presence of an active ligand. This is accomplished by the ability of the SAM riboswitch to assume one of two alternate conformations that are dictated by the level of an active ligand (McDaniel, B. A. M., Grundy, F. J., Artsimovitch, I., and Henkin, T. M. 2003 Proc. Natl. Acad. Sci. U.S.A. 100, 3083-3088, Epshtein, V., Mironov, A. S., and Nudler, E. 2003 Proc Natl Acad Sci USA 100, 5052-5056, Winkler, W. C., Nahvi, A., Sudarsan, N., Barrick J. E., and Breaker R. R. 2003 Nat. Struct. Biol. 10 701-707). X-ray crystal structural determination (Montange, R. K. and Batey, R. T. 2006 Nature 441, 1172-1175) reveals that in the presence of a saturating concentration of SAM, SAM riboswitches fold into a unique structure consisting of a conserved and primarily non base paired core. The formation of this structure via ligand binding enables the nascent RNA immediately downstream of the riboswitch to form a terminator hairpin (a stable hairpin followed by a stretch of U residues), thus inducing transcription termination (FIG. 3). If a saturating concentration of SAM is present during the synthesis of the 5′ UTR and thus SAM is bound to the SAM riboswitches, SAM riboswitches fold into a unique receptor structure comprised of four helices (P1 through P4). The formation of the base-paired structure (P1) in SAM-bound riboswitches results in the formation of an anti-anti-terminator hairpin, thus producing a terminator hairpin. This results in the induction of transcription termination. Alternatively, at subsaturating ligand concentration of SAM, however, an alternate structure dominates and form an anti-terminator hairpin structure (FIG. 3). As a result, this hairpin prevents the formation of a terminator hairpin, thereby allowing transcription of the entire mRNA.

Reagents can be obtained from sources as described above. S-(5′-Adenosyl)-L-methionine chloride (SAM, catalog #A7007) and S-(5′-Adenosyl)-L-homocysteine (SAH, catalog #A9384) may be purchased from Sigma.

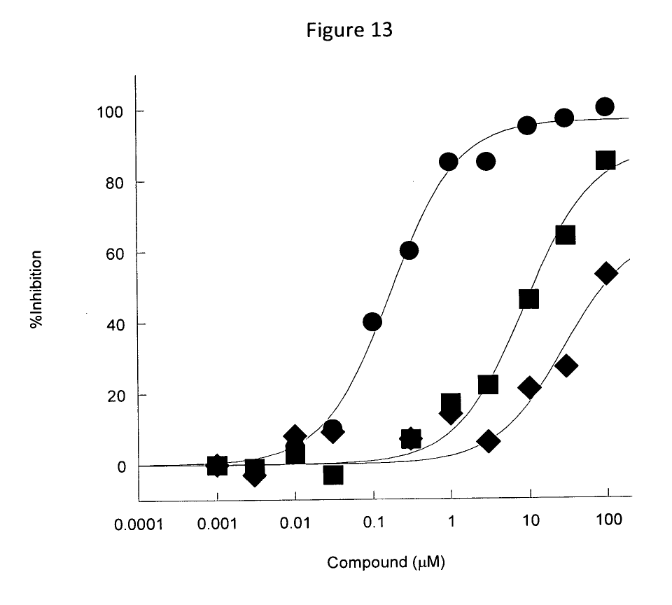

An assay using SAM and SAM analogs is depicted in FIG. 13.

### Example 13

### Assay Using a Different Detection Element is Evaluated for FMN-Riboswitch Mediated Transcription

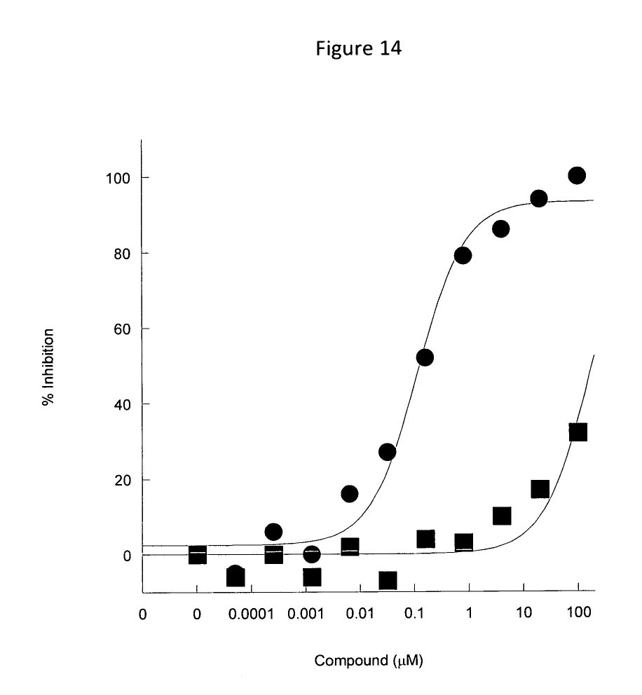

To demonstrate the utility of this assay, using a different detection element as an example, for identifying compounds with different degrees of activity that modulate FMN-riboswitch mediated transcription, the assay was performed to measure IC₅₀ values for FMN using an RNA binding fluorophore as the detection reagent (FIG. 14). In vitro transcription reactions were carried out as described above under the following conditions: 10 μM each rNTP, 3 mM Mg²⁺, 1.5 pmoles biotinylated oligo (dT), 0.15 μL DNA sequence, 0.05 Unit RNA polymerase and 0.0005% of Tween-20 replacing BSA. FMN and roseoflavin were titrated so that FMN and roseoflavine can be evaluated for their effects on FMN-riboswitch mediated transcription in a dose-dependent manner. Following a 4-hour reaction, the reactions were quenched by EDTA. The wells were then washed once with a high salt wash buffer, followed by the addition of 70 μL of RiboGreen® RNA quantitation reagent. RiboGreen® reagent is an example of an RNA binding dye which becomes highly fluorescent upon binding to RNA. After the reactions were incubated for 30 minute, reaction mixtures in the wells were excited at 480 nm and the resulting emission at 520 nm was read by SpectraMax® Multi-Mode Microplate reader. Other fluorescence readers may be used as well. FIG. 14 describes dose response curves of FMN () and roseoflavine (▪) with IC₅₀ values of 116 nM and >100,000 nM, respectively. This is an example of an induction of in vitro riboswitch-mediated transcription termination by FMN and roseoflavin, using a different detection element. This is also an example of the use of the assay to differentiate compounds with a different degree of functional activities, using a different detection element. Data are fit with KaleidaGraph software using a three parameter non-linear logistic model.

### Example 14

### Assays Using a Different Detection Element Evaluated for TPP- and SAM-Riboswitch Mediated Transcription

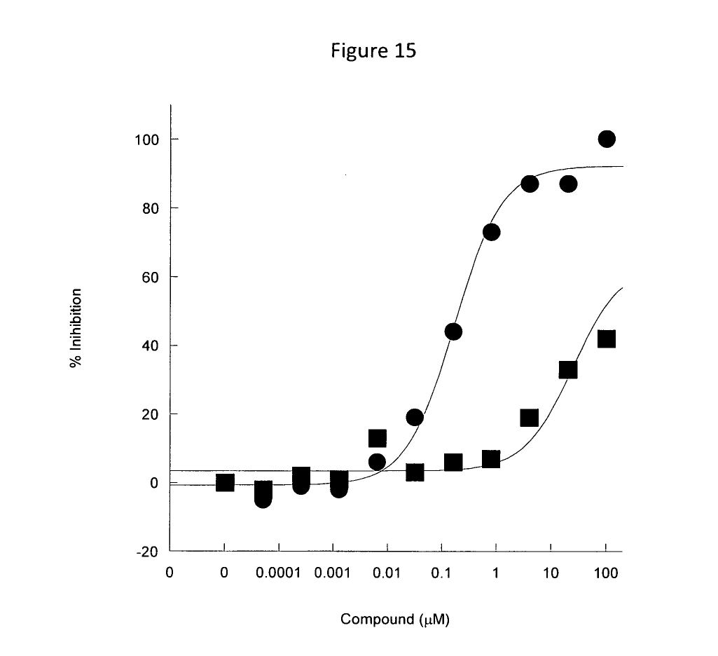

To demonstrate the utility of this assay, using a different detection element as an example, for identify compounds with different degrees of activity that modulate TPP-riboswitch mediated transcription, the assay was performed to measure IC₅₀ values for TPP and TMP using a RNA binding fluorophore as the detection reagent (FIG. 15). In vitro transcription reactions were carried out as described above under the following conditions: 5 μM each rNTP, 2 mM Mg2+, 6 pmoles biotinylated oligo (dT), 0.1 μL DNA sequence, 0.04 Unit RNA polymerase and 0.0005% of Tween-20 replacing BSA. TPP and TMP were titrated in in vitro transcription reactions to determine their effect on TPP-riboswitch mediated transcription in a dose-dependent manner. Following a 3-hour reaction, the reactions were quenched by EDTA. The wells were then washed once with a high salt wash buffer, Followed by the addition of 70 ul of RiboGreen® RNA quantitation reagent. After the reactions were incubated for 30 minute, reaction mixtures in the wells were excited at 480 nm and the resulting emission at 520 nm was read by SpectraMax® Multi-Mode Microplate reader. Other fluorescence readers may be used as well.

FIG. 15 describes a dose response curve of TPP () and TMP (▪) with IC₅₀ values of 170 nM and >25,000 nM, respectively. This is an example of an induction of in vitro riboswitch-mediated transcription termination by TPP and TMP, using a different detection element, as shown by consistency with FIG. 12. This is also an example of the use of the assay to differentiate compounds with a different degree of functional activities, using a different detection element. Data are fit with KaleidaGraph software using a three parameter non-linear logistic model.

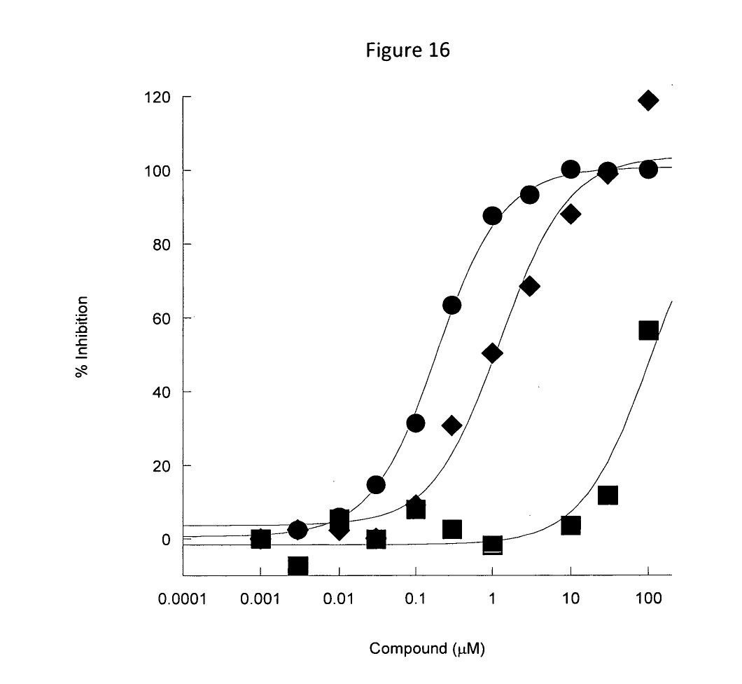

FIG. 16 describes a dose response curve of SAM () and SAM analogues. This is an example of an induction of in vitro riboswitch-mediated transcription termination by TPP and TMP, using a different detection element, as shown by consistency with FIG. 13. This is also an example of the use of the assay to differentiate compounds with a different degree of functional activities, using a different detection element. Data are fit with KaleidaGraph software using a three parameter non-linear logistic model.

Alternative combinations and variations of the examples provided will become apparent based on this disclosure. It is not possible to provide specific examples for all of the many possible combinations and variations of the embodiments described, but such combinations and variations may be claims that eventually issued.

1. A method for measuring the effect of a test compound on induction of riboswitch-mediated transcription termination, comprising:
providing a DNA sequence having a promoter operably linked to a riboswitch aptamer domain, and a riboswitch expression platform, which controls transcription of a coding region encoding a signaling sequence;
incubating, in the presence or absence of a test compound, the DNA sequence with an RNA polymerase and a plurality of ribonucleotides;
capturing the RNA product having a signaling sequence, using a capture element which specifically recognizes at least a portion of the signaling sequence; wherein the capture element is bound to a substrate;
removing uncaptured products; and
detecting the captured RNA product, wherein a decrease or absence of captured RNA product in the presence of a test compound relative to amount of captured RNA product in the absence of a test compound indicates ligand-induced riboswitch-mediated transcription termination.

2. The method of claim 1, wherein the signaling sequence is a defined nucleotide sequence and the capture element is an oligonucleotide complementary to the signaling sequence.

3. The method of claim 2, wherein the signaling sequence is a polyA sequence and the capture element is a deoxythymidylate oligonucleotide.

4. The method of claim 1, wherein at least one of the plurality of ribonucleotides is radiolabeled or fluorescence labeled.

5. The method of claim 1 wherein the captured RNA product is contacted with a labeled detection element that binds specifically to the captured RNA product.

6. The method of claim 1, wherein the riboswitch aptamer domain is selected from the aptamer domains of FMN-, TPP- or SAM-responsive riboswitches.

7. The method of claim 1, wherein the riboswitch aptamer domain is an engineered aptamer domain and the riboswitch expression platform domain is an engineered expression platform domain, which controls transcription of a coding region encoding a signaling sequence.

8. A screening kit comprising
a DNA sequence having a promoter operably linked to a riboswitch aptamer domain, and
a riboswitch expression platform, which controls transcription of a coding region encoding a signaling sequence;
RNA polymerase and a plurality of ribonucleotides;
a capture element bound to a substrate, which specifically recognizes at least a portion of a signaling sequence of an RNA transcript transcribed from the coding region.

9. (canceled)
